(19) United States
(12) Patent Application Publication
(10) Pub. No.: US 2022/0381769 A1
(43) Pub. Date: Dec. 1, 2022

(54) Immune Assay for Monitoring Response to Oncolytic Vaccinia Virus and Uses Thereof

(71) Applicants: SillaJen, Inc., Busan (KR); Regeneron Pharmaceuticals, Inc., Tarrytown, NY (US)

(72) Inventors: Nicholas GASPAR, Livermore, CA (US); Raquel DEERING, Ridgefield, CT (US); Myles DILLON, Weston, CT (US)

(73) Assignees: SillaJen, Inc., Busan (KR); Regeneron Pharmaceuticals, Inc., Tarrytown, NY (US)

(21) Appl. No.: 17/761,932

(22) PCT Filed: Sep. 23, 2020
(86) PCT No.: PCT/US2020/052222

§ 371 Date: Mar. 18, 2022

Related U.S. Application Data
(60) Provisional application No. 62/904,441, filed on Sep. 23, 2019.

(51) Int. Cl. G01N 33/50; C07K 14/005

(52) U.S. Cl. CPC G01N 33/505; C07K 14/005; G01N 2800/52

(57) ABSTRACT

Provided herein are methods and compositions for assaying vaccinia virus-specific T cell responses in a sample from a subject undergoing treatment with an oncolytic vaccinia virus. The compositions comprise custom peptide pools from known immunogenic vaccinia virus epitopes in an HLA-agnostic format to profile peripheral CD8+T cell responses.

### CROSS-REFERENCE TO RELATED APPLICATIONS

This application claims the benefit of U.S. Provisional Patent Application Ser. No. 62/904,441, filed Sep. 23, 2019, the full disclosure of which is incorporated herein by reference.

### FIELD OF THE INVENTION

The present invention relates to an HLA-agnostic immune assay based on the evaluation of cytokine-producing antigen-specific CD8+T lymphocytes responding to one or more pools of peptide sequences, each sequence corresponding to a vaccinia virus epitope. The present invention also relates to kits and compositions based on the specific epitope sequences for use in monitoring vaccinia virus specific T cell responses and identifying subjects responsive to vaccinia virus treatment.

### BACKGROUND OF THE INVENTION

Oncolytic viruses are engineered to preferentially replicate within and kill tumor cells, inducing anti-tumor immune responses. While monotherapy treatment with oncolytic viruses has shown limited clinical response in some cancers, their full immune-stimulating mechanisms have not been elucidated. A critical component to delineate the anti-tumor activity of oncolytic viruses is to monitor peripheral immune responses to both the virus and the tumor.

Traditional methods of monitoring peripheral immune response employ overlapping peptide pools that allow for assaying the entire repertoire of epitopes within a protein, regardless of HLA expression by the patient. A length of 15 amino-acids for each peptide enables tracking of both CD4⁺ and CD8⁺T cell responses. However, in order to cover the whole proteome of the virus, 1000s of peptides are necessary to screen. This quantity of peptides requires a significant volume of blood donation from each patient to successfully screen the entire peptide set, which is not feasible to attain. Additionally, while the 15 amino-acid length enables for tracking all T cells responses, it does not allow the delineation of CD8⁺ versus CD4⁺ responses.

There is a need in the art for improved assays to monitor functional CD8⁺T cell responses in the blood of patients treated with oncolytic viruses that minimize patient sample requirements in an HLA-agnostic manner.

### SUMMARY OF THE INVENTION

In the present examples, certain T-cell epitopes of vaccinia virus proteins are described and analyzed for their ability to elicit a vaccinia virus-specific CD8⁺ immune response in patients with renal cell carcinoma undergoing treatment with oncolytic vaccinia virus JX-594 (aka Pexa-Vec). Those epitopes were identified by searching the Immune Epitope Database for all curated MHC-1-restricted epitopes (n=213) and then reviewing pertinent literature to confirm restriction and remove/combine duplicates (n=72). Once identified, peptides containing those epitopes, listed at Table 3, were synthesized and sub-pooled based on MHC-1 restriction and their immunostimulatory activity in peripheral blood mononuclear cells (PBMCs) obtained from the patients at different time points during vaccinia virus therapy was assessed and correlated with disease outcome.

In several embodiments, an assay for measuring a vaccinia virus-specific T lymphocyte response in a subject is provided using one or more predetermined pools of peptides, wherein each peptide corresponds to an MCH class 1-restricted vaccinia virus epitope of Table 3, and wherein each pool comprises epitopes of one or more MHC-1 supertypes. Also provided are compositions for use in measuring an immune response against an oncolytic vaccinia virus, the compositions comprising a population of peptides comprising between 1 and 20 different peptides, wherein each peptide in the population comprises, consists of, or consists essentially of, an amino acid sequence selected from those set forth in SEQ ID NOs:1-70 (i.e. each peptide having a sequence set in forth Table 3). In some embodiments, the compositions comprise between 1 and 15, between 1 and 10, between 1 and 5, between 5 and 10, between 5 and 15, or between 5 and 20 different peptides. In related embodiments, the compositions comprise at least 5, at least 10, or at least 15 or more different peptides, wherein each peptide in the population comprises, consists of, or consists essentially of, an amino acid sequence selected from those set forth in SEQ ID NOs:1-70 (i.e. each peptide having a sequence set in forth Table 3). In some aspects, peptides have a length of 8-20 amino acids, such as 8, 9, 10, 11, 12, 13, 14, 15, 16, 17, 18, 19, or 20 amino acids. In particular aspects, the peptides have a length of 9-15 amino acids (e.g. 9 or 15 amino acids).

In preferred embodiments, each composition comprises a population of peptides, each peptide comprising, consisting essentially of, or consisting of, an epitope set forth in Table 3 having the same or similar MHC-1 class restriction.

Thus, in some preferred embodiments, a first composition is provided comprising at least 5, at least 10 or at least 15 or more different peptides, each peptide having an A*02 MHC type 1 class restricted epitope selected from SEQ ID NOs: 1-18. In related embodiments, a first composition is provided comprising a population of peptides, each peptide comprising, consisting essentially of, or consisting of, an amino acid sequence forth in SEQ ID NOs: 1-18, wherein the population of peptides comprises every epitope set forth in SEQ ID NOs:1-18.

In other preferred embodiments, a second composition is provided comprising at least 5, at least 10 or at least 15 or more different peptides, each peptide having an A*02:01 MHC type 1 class restricted epitope selected from SEQ ID NOs: 19-34. In related embodiments, a second composition is provided comprising a population of peptides, each peptide comprising, consisting essentially of or consisting of an amino acid sequence forth in SEQ ID NOs: 19-34, wherein the population of peptides comprises every epitope set forth in SEQ ID NOs:19-34.

In other preferred embodiments, a third composition is provided comprising at least 5, at least 10 or at least 15 or more different peptides, each peptide having an A*01, A*01:01, A*03, A*23:01, A*24, A*26 or A*29:03/02 MHC type 1 class restricted epitope selected from SEQ ID NOs: 35-49. In related embodiments, a third composition is provided comprising a population of peptides, each peptide comprising, consisting essentially of or consisting of an amino acid sequence forth in SEQ ID NOs: 35-49, wherein the population of peptides comprises every epitope set forth in SEQ ID NOs:35-49.

In other preferred embodiments, a fourth composition is provided comprising at least 5, at least 10 or at least 15 or more different peptides, each peptide having a B* MHC type 1 class restricted epitope selected from SEQ ID NOs: 50-70. In related embodiments, a third composition is provided comprising a population of peptides, each peptide comprising, consisting essentially of or consisting of an amino acid sequence forth in SEQ ID NOs: 50-70, wherein the population of peptides comprises every epitope set forth in SEQ ID NOs:50-70.

In some aspects, a method of assaying for vaccinia virus-specific T cells is provided comprising contacting a biological sample from a subject (e.g. whole blood or PBMC) that has been administered one or more treatments of oncolytic vaccinia virus with a first, second, third and/or fourth composition as described above, for a sufficient amount of time to cause release of a cytokine and detecting the released cytokine. In preferred aspects, a T lymphocyte response is determined by measuring a cytokine selected from IFN-γ, TNF-α, or GMCSF. In some embodiments, the biological sample is contacted with the first, second, third and fourth composition as described above.

Generally, a biological sample from a subject is either whole blood drawn from a subject or monocytes isolated from blood obtained from the subject using methods known in the art. In a preferred example, PBMCs are extracted from whole blood from a subject using, e.g., ficoll, a hydrophilic polysaccharide that separates layers of blood, and gradient centrifugation, which separates the blood into a top layer of plasma, followed by a layer of PBMCs and a bottom fraction of polymorphonuclear cells.

In some preferred embodiments, an immune response against an oncolytic vaccinia virus is determined by contacting blood or peripheral blood mononuclear cells (PBMC) from a subject that has received one or more oncolytic vaccinia virus treatments with a first, second, third and/or fourth composition as described above, for a sufficient amount of time to cause release of a cytokine and detecting the released cytokine. In particularly preferred embodiments, PBMCs obtained from a subject with cancer that has received one or more treatments with an oncolytic vaccinia virus are contacted with a plurality of peptides of Table 3 and IFN-γ is measured by enzyme-linked immunospot (ELISPOT). In related embodiments, PBMCs from the subject are contacted with a first, second, third and fourth composition as described above and IFN-γ is measured by ELISPOT.

In some embodiments, unfractionated PBMCs obtained from a subject with cancer that has received one or more treatments with an oncolytic vaccinia virus are directly stimulated with a plurality of peptides of Table 3 in the absence of exogenously added antigen presenting cells or cytokines for a period of about 5 to 50 hours and production of a cytokine (e.g. IFNγ) is measured.

In other embodiments, PBMCs obtained from a subject with cancer that has received one or more treatments with an oncolytic vaccinia virus are incubated with a plurality of peptides of Table 3 in the presence of one or more T cell supportive cytokines (e.g. IL-4, IL-7, IL-15, GM-CSF) and autologous antigen presenting cells (e.g. dendritic cells) and expanded for a period of 5 to 20 days, more preferably 8 to 15 days, most preferably about 10 days, during which production of a cytokine (e.g. IFNγ) is measured. Preferably, the T cell supportive cytokines are added substantially simultaneously and comprise IL-4, IL-7, IL-15 and GM-CSF and the PBMCs are expanded for a period of about 8 to 12 days, preferably about 10 days, during which time production of IFNγ is measured. In some aspects, the autologous dendritic cells are matured from fractionated PBMCs and added to the mixture. In other aspects, dendritic cells are already present in the PBMC sample.

In some embodiments, the subject from whom PBMCs are obtained has been treated with an oncolytic virus vaccinia virus and one or more checkpoint inhibitors. In some preferred embodiments, the one or more checkpoint inhibitors comprise a PD-1, PD-L1, and/or CTLA4 inhibitor. In some particularly preferred embodiments, the subject from whom PBMCs are obtained has been treated with a monoclonal antibody that binds to and inhibits the activity of PD-1, PD-L1 and/or CTLA4, most preferably a monoclonal antibody that binds to and inhibits the activity of PD-1 (e.g. Cemiplimab).

It is demonstrated herein that the reactivity of CD8⁺T cells against peptides of Table 3 is useful in distinguishing disease status and outcome in patients (e.g. patients with cancer) that have been administered one or more vaccinia virus treatments. Thus, in related embodiments, the assay is used to determine and/or quantify the immune responsiveness (i.e. efficacy) of a subject with cancer to oncolytic vaccinia virus treatment by contacting a blood or PBMC sample from the subject with a composition comprising a plurality of peptide sequences of Table 3 and measuring cytokine production, wherein a level of cytokine production exceeding a predetermined threshold level indicates that the patient is responding to the oncolytic vaccinia virus treatment and wherein a level of cytokine production falling below a predetermined threshold level indicates that the patient is not responding to the oncolytic vaccinia virus treatment. In some preferred embodiments, the predetermined threshold level is the baseline level prior to treatment with an oncolytic vaccinia virus. In preferred embodiments, immune responsiveness to oncolytic vaccinia virus treatment is determined by incubating a PBMC sample from a subject with a first, second, third and/or fourth composition as described above and measuring cytokine production (e.g. by ELISPOT).

In some embodiments, an immune response to vaccinia virus treatment and/or anti-tumor efficacy of vaccinia virus in a subject is identified by an increase above a baseline (e.g. pretreatment) measurement, for example at least about a 50% increase, at least about a 75% increase, at least about a 90% increase, at least about a 100% increase, at least about a 200% increase, at least about a 300% increase and at least about a 500% increase. In other embodiments, an immune response to vaccinia virus treatment and/or anti-tumor efficacy of vaccinia virus in a subject is identified by at least a 1.5-fold, at least a 2-fold, at least a 2.5-fold, at least a 3-fold, at least a 3.5-fold, at least a 4-fold, at least a 4.5-fold or at least a 5-fold increase relative to a baseline (e.g. pretreatment) value. In some aspects, the immune response to vaccinia virus treatment is assessed by measuring IFNγ production by PBMCs from a patient with cancer undergoing one or more vaccinia virus treatments following contact with a first, second, third and/or fourth compositions as herein described.

In some embodiments, an in vitro method for determining anti-tumor efficacy of vaccinia virus treatment in a subject with cancer is provided, the method comprising (a) incubating a blood or PBMC sample isolated from a subject with cancer that has been administered one or more oncolytic vaccinia virus treatments (and optionally co-administered one or more checkpoint inhibitor treatments) with a plurality of peptides sequences of Table 3; and (b) measuring T lymphocyte response. In some aspects, a T lymphocyte response is measured by (ELISPOT), FACS or ELISA. In some aspects, the response is mediated by CD8+T cells. Preferably, a T lymphocyte response is measured by ELISPOT or Flow Cytometric Analysis if the sample is a PBMC sample. In preferred embodiments, a PBMC sample from a subject with cancer is incubated (stimulated) with a first, second, third and/or fourth composition as described above for a period of time sufficient to measure an immune response by quantitative determination of cytokine (preferably IFN-γ) production by antigen-specific CD8⁺T lymphocytes using ELISPOT.

In related embodiments, a method of monitoring the number and/or status of vaccinia-virus reactive T cells in a cancer patient that has received one or more vaccinia virus treatments is provided, the method comprising determining the patient's immune reactivity to a composition comprising a population of peptides comprising at least 5, at least 10, at least 15 or at least 20 different peptides, wherein each peptide in the population comprises, consists of, or consists essentially of an amino acid sequence selected from those set forth in SEQ ID NOs:1-70. In preferred embodiments, the method comprises determining the patient's immune reactivity to a first, second, third and/or fourth composition as described above using an ELISPOT assay. The ELISPOT assay may detect a CD4⁺ and/or CD8⁺T cell response to the vaccinia virus, and preferably detects a CD8⁺T cell response to the vaccinia virus.

For any embodiment herein described, the incubation step (in which a sample from a patient that has been administered one or more vaccinia virus treatments is incubated with a population of peptides selected from those set forth at Table 3) may be from about 4 or 5 hours to 50 hours, more preferably about 5 hours to 40 hours and even more preferably about 8 to 24 or about 16 to 24 hours or a time period in between. For example, the incubation time can be from about 2 hours to about 4 hours, about 4 hours to about 8 hours, about 8 to about 16 hours, about 16 hours to about 24 hours, about 24 hours to about 36 hours, or about 36 hours to about 50 hours. In certain embodiments, the incubation step can be at least about 2 hours, at least about 4 hours, at least about 6 hours, at least about 8 hours, at least about 10 hours, at least about 12 hours, at least about 14 hours, at least about 16 hours, at least about 18 hours, at least about 20 hours, at least about 22 hours, at least about 24 hours, at least about 26 hours, at least about 28 hours, at least about 30 hours, at least about 32 hours, at least about 34 hours, at least about 36 hours, at least about 38 hours, at least about 40 hours, at least about 42 hours, at least about 44 hours, at least about 46 hours, at least about 48, or at least about 50 hours and/or no greater than about 50 hours, no greater than about 48 hours, no greater than about 46 hours, no greater than about 44 hours, no greater than about 42 hours, no greater than about 40 hours, no greater than about 38 hours, no greater than about 36 hours, no greater than about 34 hours, no greater than about 32 hours, no greater than about 30 hours, no greater than about 28 hours, no greater than about 26 hours, no greater than about 24 hours, no greater than about 22 hours, no greater than about 20 hours, no greater than about 18 hours, no greater than about 16 hours, no greater than about 14 hours, no greater than about 12 hours, no greater than about 10 hours, no greater than about 8 hours, no greater than about 6 hours, no greater than about 4 hours, or no greater than about 2 hours. In some embodiments, after an optional initial mixing step to distribute peptide antigens throughout the sample, the sample incubating is carried out without mixing further.

For any embodiment herein described, a sample collected from a subject that has received one or more vaccinia virus treatments is generally deposited into a blood collection vessel. Notwithstanding that whole undiluted blood is the preferred and most convenient sample, the present invention extends to other samples containing immune cells such as lymph fluid, cerebral, fluid, tissue fluid and respiratory fluid including nasal and pulmonary fluid.

By “whole blood” it is meant blood from a subject that has not been substantially diluted or fractionated, maintaining the ambient environment of blood for the cells as close to natural plasma conditions as practical. Thus, the addition of small volumes or dried amounts of, for example antigen, sugar or anticoagulant does not constitute dilution in accordance with the present invention, whereas addition of culture medium in excess of the blood volume constitutes dilution. A sample may also be a derivative of whole blood obtained by processing. For example, buffy coat cells or peripheral blood mononuclear cells or antigen processing cells are obtained by methods known in the art. Whole blood may also be treated to remove components such as red blood cells and/or platelets by methods known in the art. Substantial dilution would occur by the addition to the sample of more than about 40% to 50% of the original volume.

The term “PBMC” or “peripheral blood mononuclear cells” or “unfractionated PBMC”, as used herein, refers to whole PBMC, i.e. to a population of white blood cells having a round nucleus, which has not been enriched for a given sub-population. Typically, the PBMC sample may have been subjected to a selection step to contain non-adherent PBMC (which contain T cells, B cells, natural killer (NK) cells, NK T cells and DC precursors). A PBMC sample according to the invention therefore contains lymphocytes (B cells, T cells, NK cells, NKT cells). Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer, which will preferentially lyse red blood cells. Such procedures are known to the expert in the art. In some preferred embodiments, a sample from a subject having received one or more treatments with a vaccinia virus comprises PBMCs.

In some embodiments, the sample is a blood sample. Generally, blood is maintained in the presence of an anticoagulant such as heparin which may be in the container when blood is added or is added subsequently. Optionally, a simple sugar such as dextrose is contained in the container or added to the incubation mixture. In some preferred embodiments, the blood sample is a whole blood sample. In some embodiments, whole blood from a subject is collected into a container containing antigen and/or anti-coagulant, in other embodiments, antigen and/or anti-coagulant are added to the blood thereafter.

In one embodiment, the method comprises: collecting a blood sample from a subject using a capillary sampling device and introducing blood into a suitable collection vessel. In some embodiments, the capillary sampling device comprises an anticoagulant and the antigen. In other embodiments, the collection vessel or subsequent vessel comprises the antigen. In other embodiments, the collection vessel comprises a simple sugar such as dextrose or other agent that maintains the ability of the sample cells to mount a cell mediated immune response. By whatever route, the method comprises contacting the peptide with the blood sample substantially without dilution of the sample and incubating the sample with the peptide under conditions in which the shape of the sample comprises a height that has been optimized for a particular subject or subject population or sample type. In another embodiment, the method comprises incubating the sample with the agent and detecting the presence of an effector molecule or a nucleic acid molecule capable of producing an effector molecule. In an illustrative embodiment, the immune effector molecule is a cytokine such as IFN-γ.

In other embodiments, blood is collected by standard procedures into a collection vessel and transferred to sample (testing) vessels of pre-determined dimensions to ensure that a defined volume of blood is incubated with the antigen under conditions in which the shape of the sample comprises a height or volume that has been optimized for a particular subject or subject population or sample type.

In some embodiments, the blood sample incubated with one or peptides comprises a volume of less than about 1 mL of blood or more than about 2 mL of blood. In other embodiments, the 1 mL blood sample or about 1 mL blood sample during incubation does not comprise a breadth of 13 mm.

In one aspect, the present invention provides a method of performing a cell mediated immune response assay on a sample from a subject wherein said method avoids the use of needles, the method comprising collecting blood using a capillary sampling device to take small volumes of blood. In another related aspect, the invention encompasses the practice of the herein described assays including the use of small volumes of sample such as one or more samples of about 20 μl to less than but about 1 mL. In other embodiments, standard blood sampling for cellular assay techniques are employed and larger sample volumes are used, typically 1 mL to 5 mL but encompassing volumes as great as about 10 to 200 mLs or more. In a preferred embodiment, the total incubation volume of whole blood is within the range of about 50 μl to less than about 500 μl.

In some embodiments, a vaccinia virus-specific cell mediated response according to the methods herein described is measured by detecting the presence of an effector molecule or a nucleic acid molecule capable of producing an effector molecule, preferably a cytokine such as IFN-γ. Consequently, the presence or level of such an immune effector molecule includes direct and indirect data. By way of example, high levels of IFN-γ mRNA is indirect data showing increased levels of IFN-γ. Assays known in the art for assessing RNA are described for example in Sambrook, Molecular Cloning: A Laboratory Manual, 3rd Edition, CSHLP, CSH, NY, 2001 and Ausubel (Ed) Current Protocols in Molecular Biology, 5th Edition, John Wiley & Sons, Inc, N Y, 2002.

Ligands to the immune effectors are particularly useful in detecting and/or quantitating these molecules. Antibodies to the immune effector molecules are particularly useful. Techniques for the assays contemplated herein are known in the art and include, for example, sandwich assays, ELISA and, in preferred embodiments, ELISPOT. Rapid point of care immunochromatographic devices are also included. Reference to “antibodies” includes parts of antibodies, mammalianized (e.g. humanized) antibodies, recombinant or synthetic antibodies and hybrid and single chain antibodies.

Both polyclonal and monoclonal antibodies are obtainable by immunization with the immune effectors or antigenic fragments thereof and either type is utilizable for immunoassays. The methods of obtaining both types of sera are well known in the art. Polyclonal sera are less preferred but are relatively easily prepared by injection of a suitable laboratory animal with an effective amount of the immune effector, or antigenic part thereof, collecting serum from the animal and isolating specific sera by any of the known immunoadsorbent techniques. Although antibodies produced by this method are utilizable in virtually any type of immunoassay, they are generally less favored because of the potential heterogeneity of the product.

The use of monoclonal antibodies in an immunoassay is particularly preferred because of the ability to produce them in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art.

Another aspect of the present invention contemplates, therefore, a method for detecting an immune effector in a sample comprising immune cells from a subject, said method comprising contacting said sample or an aliquot of said sample with an antibody specific for said immune effector or antigenic fragment thereof for a time and under conditions sufficient for an antibody-effector complex to form, and then detecting said complex.

A sample includes whole blood. This method includes micro-arrays and macro-arrays on planar or spherical solid supports.

A wide range of immunoassay techniques are available as can be seen by reference to U.S. Pat. Nos. 4,016,043, 4,424,279 and 4,018,653.

T-cell phenotypes may be evaluated using well known methods, e.g., T cell activation may be determined, e.g., by measuring changes in the level of expression of cytokines and/or T cell activation markers, and/or the induction of antigen-specific proliferating cells. Techniques known to those of skill in the art, including, but not limited to, immunoprecipitation followed by Western blot analysis, ELISAs, flow cytometry, Northern blot analysis, and RT-PCR can be used to measure the expression cytokines and T cell activation markers. Cytokine release may be measured by measuring secretion of cytokines including but not limited to Interleukin-2 (IL-2), Interleukin-4 (IL-4), Interleukin-6 (IL-6), Interleukin-12 (IL-12), Interleukin-16 (IL-16), PDGF, TGF-α, TGF-β, TNF-α, TNF-β, GCSF, GM-CSF, MCSF, IFN-α, IFN-0, IFN-γ, TFN-γ, IGF-I, and IGF-II (see, e.g., Isaacs et al., 2001, Rheumatology, 40: 724-738; Soubrane et al., 1993, Blood, 81(1): 15-19).

T cell modulation may also be evaluated by measuring, e.g., proliferation by, e.g., ³H-thymidine incorporation, trypan blue cell counts, and fluorescence activated cell sorting (FACS).

Although the illustrated immune effector molecule is IFN-γ, other cytokines such as TNFα and GM-CSF can be readily assayed, as are components of the complement system, perforins, defensins, cathelicidins, granzymes, Fas ligand, CD-40 ligand, exotaxin, cytotoxins, chemokines or monokines. In some embodiments the immune cells tested are selected from a natural killer (NK) cell, T-cell, B-cell, macrophage or monocyte.

In specific embodiments, the response is utilized to identify a critical level of T cell activity, including a level that can be used to determine responders vs. non-responders. In certain embodiments, a quantitative biological response is the production (such as secreted from the cell) or expression (such as membrane-bound on the cell) of a compound from T cells upon recognition of a particular target. In specific embodiments, the compound is IL1α, IL1β, IL-2, IL-3, IL-4, IL-5, IL-6, IL-9, IL-10, IL-12 (p35+p40), IL-13, IL-14, IL-15, IL-16, IL-17a, IL-17B, IL-17F, IL-18, IL-19, IL-20, IL-21, IL-22, IL-23 (p90+p43), IL-25, IL-26, IL-27 (p28+EBI3), IL-28A/B/IL29A, IL-30 (p28 subunit of IL-27), IL-31, IL-32, IL-33, IL-35 (p35+EBI3), TNFα, LTα, LTβ, LIGHT, OX40L, CD25, CD45, CD40L, FASL, CD27L, CD30L, 4-1BBL, TRAIL, RANK Ligand, GM-CSF, IFNγ, LIF, MIF, TGFβ1, TGFβ2, and/or TGFβ3. In specific embodiments, the compound is IL2, IFNγ, granzyme B, CD25, perforin, GM-CSF, and/or TNFα. In some embodiments, the biological response is the upregulation of one or more markers upon stimulation with a cognate antigen, such as CD25, CD27, CD28, CD45, CD69, and/or CD107.

In specific embodiments, the parameters for defining a threshold of activity include determination of a level of unspecific responses by measuring the magnitude of biological activity against an irrelevant target (antigens not expressed by the vaccinia virus). This level of unspecific response then establishes a baseline to discriminate between responders and non-responders. In at least some cases, the efficacy encompasses the percentage of individuals in a population that have a biological response that exceeds the threshold quantity. T cells that have greater magnitudes of response than the threshold are considered responders, whereas those with response values less than the threshold value are considered non-responders.

In the context of measuring cytotoxicity for the cells, the cytotoxicity may be determined by standard killing assays including co-culture assays, non-radioactive and radioactive labeling of antigen-loaded/expressing targets. One specific embodiment includes a chromium release assay where T cells are utilized as effectors and the targets are chromium-labeled autologous PHA blasts pulsed with the peptide(s) in question. Effector to target ratios may be (or may be at least or no more than): 5:1, 10:1, 15:1, 20:1, 25:1, 30:1, 35:1, 40:1, 45:1, 50:1, 55:1; 60:1, 65:1, 70:1, 75:1, 80:1, 85:1, 90:1, or 95:1, etc. The cells may be considered to be cytotoxic against a particular peptide if the percentage of specific lysis of PHA blasts pulsed with the relevant peptides is higher than the lysis mediated by an irrelevant T cell product or unmanipulated PBMCs. In some conditions a threshold of activity in this assay can be considered as 10% specific lysis at a 40:1 effector to target ratio (for example, after subtracting the percentage of unspecific killing of target cells expressing an unrelated peptide (e.g., not present in the pathogen or tumor of interest).

Clinical efficacy for a particular plurality of antigen-specific T cells may be determined mathematically as a function of antigen specificity and/or cytotoxicity. In specific embodiments, a quantitative value for the efficacy of the cells is obtained through a mathematical relationship that utilizes values for biological measurements for the antigen-specific T cells and unmanipulated control cells, such as unmanipulated PBMCs and utilizes a value for specific killing.

If added in a soluble form, the peptide(s) may be added at a concentration from about 0.1 nM to about 100 μM for each peptide. In certain embodiments, the soluble peptide(s) may be added at a concentration of about 1 nM to about 90 μM, about 10 nM to about 80 μM, about 50 nM to about 70 μM, about 100 nM to about 60 μM, about 150 nM to about 50 μM, about 200 nM to about 40 μM, about 250 nM to about 30 μM, about 300 nM to about 20 M, about 350 nM to about 10 μM, about 400 nM to about 9 μM, about 450 nM to about 8 μM, about 500 nM to about 7 μM, about 550 nM to about 6 μM, about 600 nM to about 5 M, about 650 nM to about 4 μM, about 700 nM to about 3 μM, about 750 nM to about 2.5 μM, about 800 nM to about 2 μM, about 900 nM to about 1.5 μM, or about 950 nM to about 1.25 μM for each peptide. In certain embodiments, the soluble peptide(s) may be added at a concentration of about 100 nM to about 100 μM, about 250 nM to about 75 μM, about 500 nM to about 50 μM, about 750 nM to about 25 μM, about 900 nM to about 10 μM or about 990 nM to about 5 μM for each peptide.

In certain embodiments, the soluble peptide(s) may be added at a concentration of at least about 0.1 nM, about 1 nM, about 5 nM, about 10 nM, about 20 nM, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 260 nM, about 270 nM, about 280 nM, about 290 nM, about 300 nM, about 310 nM, about 320 nM, about 330 nM, about 340 nM, about 350 nM, about 360 nM, about 370 nM, about 380 nM, about 390 nM, about 400 nM, about 410 nM, about 420 nM, about 430 nM, about 440 nM, about 450 nM, about 460 nM, about 470 nM, about 480 nM, about 490 nM, about 500 nM, about 510 nM, about 520 nM, about 530 nM, about 540 nM, about 550 nM, about 560 nM, about 570 nM, about 580 nM, about 590 nM, about 600 nM, about 610 nM, about 620 nM, about 630 nM, about 640 nM, about 650 nM, about 660 nM, about 670 nM, about 680 nM, about 690 nM, about 700 nM, about 710 nM, about 720 nM, about 730 nM, about 740 nM, about 750 nM, about 760 nM, about 770 nM, about 780 nM, about 790 nM, about 800 nM, about 810 nM, about 820 nM, about 830 nM, about 840 nM, about 850 nM, about 860 nM, about 870 nM, about 880 nM, about 890 nM, about 900 nM, about 910 nM, about 920 nM, about 930 nM, about 940 nM, about 950 nM, about 960 nM, about 970 nM, about 980 nM, about 990 nM, about 1 μM, about 2 μM, about 3 μM, about 4 μM, about 5 μM, about 6 μM, about 7 μM, about 8 μM, about 9 μM, about 10 μM, about 20 M, about 30 μM, about 40 μM, about 50 μM, about 60 μM, about 70 μM, about 80 M, about 90 μM, or about 100 μM for each peptide.

In certain embodiments, the soluble peptide(s) may be added at a concentration of about 0.1 nM, about 1 nM, about 5 nM, about 10 nM, about 20 nM, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 260 nM, about 270 nM, about 280 nM, about 290 nM, about 300 nM, about 310 nM, about 320 nM, about 330 nM, about 340 nM, about 350 nM, about 360 nM, about 370 nM, about 380 nM, about 390 nM, about 400 nM, about 410 nM, about 420 nM, about 430 nM, about 440 nM, about 450 nM, about 460 nM, about 470 nM, about 480 nM, about 490 nM, about 500 nM, about 510 nM, about 520 nM, about 530 nM, about 540 nM, about 550 nM, about 560 nM, about 570 nM, about 580 nM, about 590 nM, about 600 nM, about 610 nM, about 620 nM, about 630 nM, about 640 nM, about 650 nM, about 660 nM, about 670 nM, about 680 nM, about 690 nM, about 700 nM, about 710 nM, about 720 nM, about 730 nM, about 740 nM, about 750 nM, about 760 nM, about 770 nM, about 780 nM, about 790 nM, about 800 nM, about 810 nM, about 820 nM, about 830 nM, about 840 nM, about 850 nM, about 860 nM, about 870 nM, about 880 nM, about 890 nM, about 900 nM, about 910 nM, about 920 nM, about 930 nM, about 940 nM, about 950 nM, about 960 nM, about 970 nM, about 980 nM, about 990 nM, about 1 M, about 2 μM, about 3 M, about 4 μM, about 5 μM, about 6 μM, about 7 μM, about 8 M, about 9 μM, about 10 μM, about 20 M, about 30 μM, about 40 μM, about 50 μM, about 60 μM, about 70 μM, about 80 μM, about 90 μM, or about 100 μM for each peptide. In certain embodiments, the soluble peptide(s) may be added at a concentration of about 1 μM for each peptide.

The ratio of cells in the sample (e.g., whole blood sample) to the peptide(s) is about 1:1 to about 1:100. In certain embodiments, the ratio of cells in the sample to peptide(s) is about 1:1 to about 1:90; about 1:1 to about 1:80, about 1:1 to about 1:70, about 1:1 to about 1:60, about 1:1 to about 1:50, about 1:1 to about 1:40, about 1:1 to about 1:30, about 1:1 to about 1:20, about 1:1 to about 1:10, about 1:1 to about 1:9, about 1:1 to about 1:8, about 1:1 to about 1:7, about 1:1 to about 1:6, about 1:1 to about 1:5, about 1:1 to about 1:4, about 1:1 to about 1:3, or about 1:1 to about 1:2. In certain embodiments, the ratio of cells in the peptide(s) is about 1:2 to about 1:90; about 1:3 to about 1:80, about 1:4 to about 1:70, about 1:5 to about 1:60, about 1:6 to about 1:50, about 1:7 to about 1:40, about 1:8 to about 1:30, or about 1:9 to about 1:20.

In certain embodiments, the ratio of cells in the sample to peptide(s) is at least about 1:2, about 1:3, about 1:4, about 1:5, about 1:6, about 1:7, about 1:8, or about 1:9, or about 1:10, or about 1:12, or about 1:14, or about 1:16, or about 1:18, or about 1:20, or about 1:25, or about 1:30, or about 1:35, or about 1:40, or about 1:45, or about 1:50, or about 1:55, or about 1:60, or about 1:65, or about 1:70, or about 1:75, or about 1:80, or about 1:85, or about 1:90, or about 1:100.

In certain embodiments, the ratio of cells in the sample to peptide(s) is about 1:2, about 1:3, about 1:4, about 1:5, about 1:6, about 1:7, about 1:8, or about 1:9, or about 1:10, or about 1:12, or about 1:14, or about 1:16, or about 1:18, or about 1:20, or about 1:25, or about 1:30, or about 1:35, or about 1:40, or about 1:45, or about 1:50, or about 1:55, or about 1:60, or about 1:65, or about 1:70, or about 1:75, or about 1:80, or about 1:85, or about 1:90, or about 1:100.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

The term “T-cell,” as used herein, refers to all types of immune cells expressing CD3, including CD4+ cells (helper T cells), CD8+ cells (cytotoxic T cells), regulatory T cells (Tregs), natural killer cells, and tumor infiltrating lymphocytes.

Any of the peptide compositions described herein may be comprised in a kit. In a non-limiting example, sample extraction apparatuses, cell culture media, cell culture flasks, peptides, viruses, and/or other reagents may be comprised in a kit.

The kits may comprise suitably aliquoted compositions utilized in the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the compositions in close confinement for commercial sale. Such containers may include injection or blow-molded plastic containers into which the desired vials are retained. In specific embodiments, the kit comprises one or more means for obtaining blood cells and/or bone marrow cells. In specific embodiments, the kit comprises instructions for use.

When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means.

The methods described herein may be used to assay a patient's immune responsiveness to any oncolytic vaccinia virus, whether wild type or genetically modified. In preferred embodiments, the oncolytic vaccinia virus is a wild type or modified Copenhagen, Western Reserve, Lister or Wyeth strain. The genome of the Western Reserve vaccinia strain has been sequenced (Accession number AY243312).

In some embodiments, the oncolytic vaccinia virus is modified to increase cancer selectivity. By way of example, the replicative oncolytic vaccinia virus may be engineered to lack one or more functional genes in order to increase the cancer selectivity of the virus. In some preferred embodiments, the oncolytic vaccinia virus is engineered to lack thymidine kinase (TK) activity. A TK-deficient vaccinia virus requires thymidine triphosphate for DNA synthesis, which leads to preferential replication in dividing cells (particularly cancer cells). In another aspect, the oncolytic vaccinia virus may be engineered to lack vaccinia virus growth factor (VGF). This secreted protein is produced early in the infection process, acting as a mitogen to prime surrounding cells for infection. In another aspect, the oncolytic vaccinia virus may be engineered to lack both VFG and TK activity. In other aspects, the oncolytic vaccinia virus may be engineered to lack one or more genes involved in evading host interferon (IFN) response such as E3L, K3L, B18R, or B8R. In some preferred embodiments, the replicative oncolytic vaccinia virus is a Western Reserve, Copenhagen, Lister or Wyeth strain and lacks a functional TK gene. In other embodiments, the oncolytic vaccinia virus is a Western Reserve, Copenhagen, Lister or Wyeth strain lacking a functional B18R and/or B8R gene.

The oncolytic virus may be administered by any administration route. In certain embodiments the virus is administered intravascularly, intratumorally, or a combination thereof. In a further aspect administration is by injection into a tumor mass. In still a further embodiment, administration is by injection into or in the region of tumor vasculature. In yet a further embodiment, administration is by injection into the lymphatic or vasculature system regional to said tumor. In some embodiments, the subject has metastatic cancer.

A subject's immune response to a vaccinia virus may be measured at any time point during an oncolytic vaccinia virus treatment regimen. For example, a blood or PBMC sample from a cancer patient may be obtained after a first, second, third, fourth, fifth, sixth, or subsequent administration of vaccinia virus and contacted with a first, second, third and/or fourth composition as herein described. Preferably, a blood or PBMC sample from the patient is also obtained just prior to a first administration of oncolytic vaccinia virus as a baseline measurement.

In some aspects, the oncolytic virus is administered intravenously and/or intratumorally to a subject in two or more (e.g. 3, 4, 5, 6, 7, 8, 9, 10 or more) weekly doses of about 10⁷-10¹¹ plaque forming units (pfu), preferably about 10⁹ pfu, which may be followed by less frequent dosing (e.g. every other week, every three weeks, or monthly). The subject's immune response to the vaccinia virus may be measured at baseline and 1, 2, 3, 4, 5, 6, 7, 8, 9, 10, 11, 12, 13, 14, 15, or 16 or more weeks after a first dose of vaccinia virus is administered. In some embodiments, the methods described herein comprise incubating a blood or PBMC sample obtained from the patient about four to 16 weeks (e.g. six weeks and/or twelve weeks) after a first dose of vaccinia virus is administered with a first, second, third and/or fourth composition as herein described.

In an embodiment, a subject administered an oncolytic vaccinia virus has a cancer selected from brain cancer (e.g. glioblastoma), renal cancer, liver cancer, lung cancer, head and neck cancer, breast cancer, pancreatic cancer, prostate cancer, bone cancer, testicular cancer, cervical cancer, ovarian cancer, uterine cancer, rectal cancer, gastrointestinal cancer, lymphoma, pre-neoplastic lesions in the lung, colon cancer, melanoma, and bladder cancer. In particularly preferred embodiments, the subject has a cancer selected from renal cell carcinoma, hepatocellular carcinoma and melanoma.

Also provided is a kit for assaying an immune response against a vaccinia virus, the kit comprising a peptide composition comprising at least 5, at least 10, at least 15 or at least 20 or more different peptides, wherein each peptide in the population comprises, consists of, or consists essentially of an amino acid sequence selected from those set forth in SEQ ID NOs:1-70 (i.e. each peptide having a sequence set in forth Table 3). In some aspects, the kit comprises a first, second, third and/or fourth composition as described above.

Some embodiments of the invention are exemplified in the following items 1 to 58:

1. A composition comprising a population of peptides comprising at least 5, at least 10, at least 15 or at least 20 or more different peptides, wherein each peptide in the population comprises, consists of, or consists essentially of an amino acid sequence selected from those set forth in SEQ ID NOs:1-70.

2. A composition according to item 1, comprising a population of peptides, each peptide comprising, consisting essentially of, or consisting of an amino acid sequence forth in SEQ ID NOs: 1-18, preferably wherein the population of peptides comprises the epitopes set forth in SEQ ID NOs:1-18.

3. A composition according to item 1, comprising a population of peptides, each peptide comprising, consisting essentially of, or consisting of an amino acid sequence forth in SEQ ID NOs: 19-34, preferably wherein the population of peptides comprises the epitopes set forth in SEQ ID NOs:19-34.

4. A composition according to item 1, comprising a population of peptides, each peptide comprising, consisting essentially of, or consisting of an amino acid sequence forth in SEQ ID NOs: 35-49, preferably wherein the population of peptides comprises the epitopes set forth in SEQ ID NOs:35-49.

5. A composition according to item 1, comprising a population of peptides, each peptide comprising, consisting essentially of, or consisting of an amino acid sequence forth in SEQ ID NOs: 50-70, preferably wherein the population of peptides comprises the epitopes set forth in SEQ ID NOs:50-70.

6. A method for monitoring the number and/or status of vaccinia virus-reactive T cells, preferably CD8⁺T cells, in a patient that has received one or more oncolytic vaccinia virus treatments, the method comprising determining the patient's immune reactivity to a composition according to item 1.

7. The method of item 6, comprising contacting a PBMC or whole blood sample from the patient with one or more compositions according to item 1.

8. The method of item 6, wherein the patient's immune reactivity to the one or more compositions is assessed by ELISPOT assay.

9. The method of item 8, wherein the ELISPOT assay quantifies the level of one or more cytokines produced by CD8⁺T cells in response to the one or more compositions.

10. The method of item 9, wherein the one or more cytokines include IFN-γ.

11. The method of item 7, wherein the sample obtained from the patient is incubated with the one or more compositions for a period of 5 to 50 hours.

12. The method of item 11, wherein the sample obtained from the patient is incubated with the one or more compositions for a period of 36 to 50 hours.

13. The method according to item 6, wherein the patient has a cancer selected from brain cancer, renal cancer, liver cancer, lung cancer, head and neck cancer, breast cancer, pancreatic cancer, prostate cancer, bone cancer, testicular cancer, cervical cancer, ovarian cancer, uterine cancer, rectal cancer, gastrointestinal cancer, lymphoma, pre-neoplastic lesions in the lung, colon cancer, melanoma, and bladder cancer.

14. The method according to item 13, wherein the patient has metastatic renal cell carcinoma.

15. The method according to item 6, wherein the patient received one or more intratumoral and/or intravascular treatments of a wild type or genetically modified Wyeth or Western Reserve strain vaccinia virus.

16. The method according to item 15, wherein the patient received one or more treatments of a genetically modified Wyeth or Western Reserve strain vaccinia virus.

17. The method according to item 16, wherein the Wyeth or Western Reserve strain vaccinia virus lacked a functional thymidine kinase gene and/or lacked a functional vaccinia growth factor gene.

18. The method according to item 16, wherein the Wyeth or Western Reserve strain vaccinia virus contained a transgene encoding a cytokine, preferably a transgene encoding GM-CSF.

19. The method according to item 18, wherein the patient received one or more treatments of JX-594.

20. The method according to item 6, wherein the vaccinia virus was administered to the patient at a dose of about 10⁶ to 10¹² pfu.

21. A method for determining the prognosis of a cancer patient that has received one or more oncolytic vaccinia virus treatments, the method comprising measuring the patient's immune reactivity to a composition according to item 1 and predicting the outcome of the cancer in said patient.

22. The method of item 21, wherein a patient having an increased immune reactivity to said composition compared to a control value indicates a long-term survival of the patient.

23. The method of item 22, wherein the control value is determined by measuring the patient's immune reactivity to a composition according to item 1 prior to receiving one or more oncolytic vaccinia virus treatments.

24. The method of item 21, wherein a patient that does not have an increased immune reactivity to said composition compared to a control value indicates a short-term survival of the patient.

25. The method of item 21, comprising contacting a PBMC or whole blood sample from the patient with one or more compositions according to item 1.

26. The method of item 21, wherein the patient's immune reactivity to the one or more compositions is assessed by ELISPOT assay.

27. The method of item 26, wherein the ELISPOT assay quantifies the level of one or more cytokines produced by CD8+T cells in response to the one or more compositions.

28. The method of item 27, wherein the one or more cytokines include IFN-γ.

29. The method of item 25, wherein the sample obtained from the patient is incubated with the one or more compositions for a period of 5 to 50 hours.

30. The method of item 29, wherein the sample obtained from the patient is incubated with the one or more compositions for a period of 36 to 50 hours.

31. The method according to item 21, wherein the patient has a cancer selected from brain cancer, renal cancer, liver cancer, lung cancer, head and neck cancer, breast cancer, pancreatic cancer, prostate cancer, bone cancer, testicular cancer, cervical cancer, ovarian cancer, uterine cancer, rectal cancer, gastrointestinal cancer, lymphoma, pre-neoplastic lesions in the lung, colon cancer, melanoma, and bladder cancer.

32. The method according to item 31, wherein the patient has metastatic renal cell carcinoma.

33. The method according to item 21, wherein the patient received one or more intratumoral and/or intravascular treatments of a wild type or genetically modified Wyeth or Western Reserve strain vaccinia virus.

34. The method according to item 33, wherein the patient received one or more treatments of a genetically modified Wyeth or Western Reserve strain vaccinia virus.

35. The method according to item 34, wherein the Wyeth or Western Reserve strain vaccinia virus lacked a functional thymidine kinase gene and/or lacked a functional vaccinia growth factor gene.

36. The method according to item 34, wherein the Wyeth or Western Reserve strain vaccinia virus contained a transgene encoding a cytokine, preferably a transgene encoding GM-CSF.

37. The method according to item 36, wherein the patient received one or more treatments of JX-594.

38. The method according to item 21, wherein the vaccinia virus was administered to the patient at a dose of about 10⁶ to 10¹² pfu.

39. A method for monitoring the efficacy of vaccinia virus treatment in a cancer patient that has been administered one or more doses of oncolytic vaccinia virus, comprising measuring the patient's immune reactivity to a composition according to item 1 and determining the efficacy of the vaccinia virus treatment in said patient.

40. The method of item 39, wherein a patient having an increased immune reactivity to said composition compared to a control value indicates that the vaccinia virus treatment is effective.

41. The method of item 40, wherein the control value is determined by measuring the patient's immune reactivity to a composition according to item 1 prior to receiving one or more oncolytic vaccinia virus treatments.

42. The method of item 39, wherein a patient that does not have an increased immune reactivity to said composition compared to a control value indicates a short-term survival of the patient.

43. The method of item 39, comprising contacting a PBMC or whole blood sample from the patient with one or more compositions according to item 1.

44. The method of item 39, wherein the patient's immune reactivity to the one or more compositions is assessed by ELISPOT assay.

45. The method of item 44, wherein the ELISPOT assay quantifies the level of one or more cytokines produced by CD8+T cells in response to the one or more compositions.

46. The method of item 45, wherein the one or more cytokines include IFN-γ.

47. The method of item 43, wherein the sample obtained from the patient is incubated with the one or more compositions for a period of 5 to 50 hours.

48. The method of item 47, wherein the sample obtained from the patient is incubated with the one or more compositions for a period of 36 to 50 hours.

49. The method according to item 39, wherein the patient has a cancer selected from brain cancer, renal cancer, liver cancer, lung cancer, head and neck cancer, breast cancer, pancreatic cancer, prostate cancer, bone cancer, testicular cancer, cervical cancer, ovarian cancer, uterine cancer, rectal cancer, gastrointestinal cancer, lymphoma, pre-neoplastic lesions in the lung, colon cancer, melanoma, and bladder cancer.

50. The method according to item 49, wherein the patient has metastatic renal cell carcinoma.

52. The method according to item 39, wherein the patient received one or more intratumoral and/or intravascular treatments of a wild type or genetically modified Wyeth or Western Reserve strain vaccinia virus.

53. The method according to item 52, wherein the patient received one or more treatments of a genetically modified Wyeth or Western Reserve strain vaccinia virus.

54. The method according to item 53, wherein the Wyeth or Western Reserve strain vaccinia virus lacked a functional thymidine kinase gene and/or lacked a functional vaccinia growth factor gene.

55. The method according to item 53, wherein the Wyeth or Western Reserve strain vaccinia virus contained a transgene encoding a cytokine, preferably a transgene encoding GM-CSF.

56. The method according to item 55, wherein the patient received one or more treatments of JX-594.

57. The method according to item 39, wherein the vaccinia virus was administered to the patient at a dose of about 10⁶ to 10¹² pfu.

58. A kit for use in measuring an immune response against an oncolytic vaccinia virus comprising one or more compositions according to items 2-5.

These and other aspects of the present invention will be apparent to those of ordinary skill in the art in the following description, claims and drawings.

### DESCRIPTION OF THE DRAWINGS

FIG. 1 illustrates the genetic modifications made to Wyeth strain vaccinia virus to create PEXA-VEC (JX-594). Briefly, lacZ and GM-CSF genes were inserted into the native thymidine kinase gene. The resulting virus expresses GM-CSF to drive active immunity and is thymidine kinase deficient, increasing selectivity for cancer cells.

FIGS. 2A-2B illustrate the administration scheme of JX-594 to patients with renal cell carcinoma and collection of peripheral blood mononuclear cells (FIG. 2A) and the clinical response of these patients (FIG. 2B).

FIG. 3 compares traditional overlapping peptide pools to the epitope-based peptide pools of the present invention. By using only peptides found in the Immune Epitope Database, the present invention ensures that each peptide included in the pools has been validated as immune-reactive in humans and enables screening of the full set with each blood collection. In contrast, 1000s of peptides would need to be screened, covering the whole proteome of the virus by the use of traditional overlapping peptide pools, which in turn requires a significant (and infeasible) volume of blood to be drawn from each patient to successfully screen the entire peptide set. Additionally, traditional overlapping peptide pools does not allow for differentiation of CD8+ versus CD4+ responses.

FIG. 4 illustrates ELISPOT IFNγ response for PBMCs collected from patients with progressive disease (n=4) (left panel), patients with stable disease (n=12) (middle panel) and a patient with complete response (n=1) (right panel) at baseline, six weeks and 12 weeks, incubated with overlapping 15mer amino-acid peptides overlapping by 11 amino acid for coverage of the second half of major core Vaccinia protein P4a.

FIGS. 5A-5B FIG. 5A illustrates the procedure for measuring ELISPOT IFNγ response for PBMCs incubated with peptide pools. FIG. 5B illustrates the clinical response of each patient from whom PBMCs were collected and assigns a symbol to each patient/outcome corresponding to those used in FIGS. 6A and 6B.

FIGS. 6A-6B compares illustrates ELISPOT IFNγ response for PBMCs collected at the indicated time points from patients with progressive disease (n=4), patients with stable disease (n=12) and a patient with complete response (n=1) to overlapping versus epitope-based peptide pools (FIG. 6A) and illustrates ELISPOT IFNγ response for PBMCs collected at the indicated time points from patients with progressive disease (n=4), patients with stable disease (n=12) and a patient with complete response (n=1), incubated with epitope-based peptide pools according to Table 3 (FIG. 6B).

FIG. 7 illustrates ELISPOT IFNγ response for PBMCs

FIGS. 8A-8B FIG. 8A illustrates the fold-change over time in IFNγ response of PBMCs to the epitope-based peptide pools according to Table 3 compared to baseline. Three out of four patients with progressive disease exhibited a fold-change below baseline whereas 8/11 patients with stable or complete response exhibited a fold-change above baseline. FIG. 8B illustrates that the same analysis performed on the response to the overlapping peptide pool did not show the same grouping of inductions as the epitope-based pools. Rather, the change in response did not trend with any specific clinical response.

FIGS. 9A-9B illustrate correlation of the fold-change in peripheral T cell response at 6 weeks to overall survival of RCC patients treated with PEXA-VEC. The change in response at week 6 for all patients in the study was analyzed in comparison to clinical response. Radiographic change was determined by calculating percent change in tumor size (mm) of the primary lesion at week 6 compared to the size at baseline. FIG. 9A illustrates overall survival correlation to T cell response to all epitope-based peptide pools. FIG. 9B illustrates the same correlation with only A*02 supertype pool.

FIG. 10 illustrates the clinical response (tumor response and % change in tumor burden) of patients with advanced RCC who received combination therapy with JX-594 and Cemiplimab (the patients had received 6 to 15 rounds of Cemiplimab at the time of data collection).

FIG. 11 illustrates the percentage of tumor change (volume) in individual patients treated with JX-594+Cemiplimab over the course of treatment.

FIG. 12 illustrates correlation between T cell response (ELISPOT) and tumor volume change in patients treated with JX-594+Cemiplimab. The left panel illustrates the results of direct ex vivo stimulation of PBMCs from the patients with the OV peptides (without expansion) and the right panel illustrates the results of expansion of PBMCs from the patients with OV peptides in the presence of autologous dendritic cells and T cell supportive cytokines for 10 days.

### DETAILED DESCRIPTION OF THE INVENTION

### Definitions

An “immune response” refers to a change in immunity, for example, a response of a cell of the immune system, such as a B cell, T cell or monocyte, to a stimulus (e.g. a response specific for a vaccinia virus antigen). In one example, an immune response is a T cell response, such as a CD4⁺ response or a CD8⁺ response.

“Measuring an immune response” refers to any measurement or determination of the level, presence or absence, reduction, or increase in an immune response in vitro or in vivo.

“Epitope”, also known as an antigenic determinant, is the part of an antigen that is recognized by the immune system, specifically by, for example, antibodies, B cells, or T cells. As used herein, “MHC Class I-restricted epitopes” are epitopes that are presented to immune cells by MHC class I molecules found on nucleated cells. In some embodiments, the epitope itself is an antigen. The T-cell epitopes presented by major histocompatibility complex class I (MHC I) molecules are CD8⁺T-cell epitopes, which are typically peptides 8-11 amino acids in length.

“Interferon-gamma (IFN- or IFNγ)” refers to a protein produced by T lymphocytes in response to a specific antigen or mitogenic stimulation. Sequences for IFN-γ are publicly available (exemplary IFN-γ protein sequences are available from GenBank Accession Nos: CAA00226; AAA72254; and 0809316A).

The term “subject” or “patient” refers to either a human or non-human, such as primates, mammals and vertebrates. In particular embodiments, the subject or patient is a human. In some embodiments, the subject or patient is a human with a cancer that is refractory to one or more standard treatments.

### EXAMPLES

The following examples illustrate preferred embodiments of the present invention and are not intended to limit the scope of the invention in any way. While this invention has been described in relation to its preferred embodiments, various modifications thereof will be apparent to one skilled in the art from reading this application.

### Example 1

Clinical Trial Design

A phase 2 clinical trial in the clear cell variant of metastatic renal cell carcinoma was conducted. It was a single-center/investigator-initiated trial at Pusan National University Yangsan Hospital in South Korea. 17 patients (median age 62, range 37-73; 12 male and 5 female) were enrolled, all of which were refractory to standard therapy. All patients had received at least one tyrosine kinase inhibitor or anti-angiogenic and most had also received at least one mTOR inhibitor (n=14) or at least one immunotherapeutic (n=10).

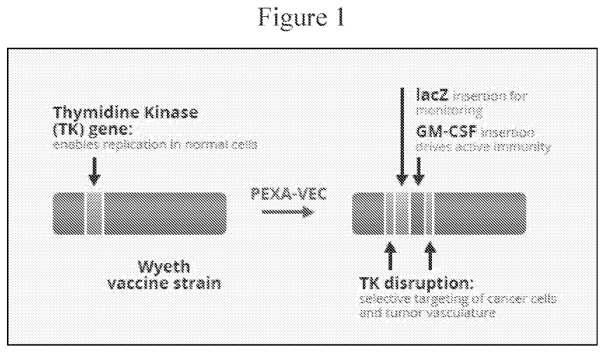

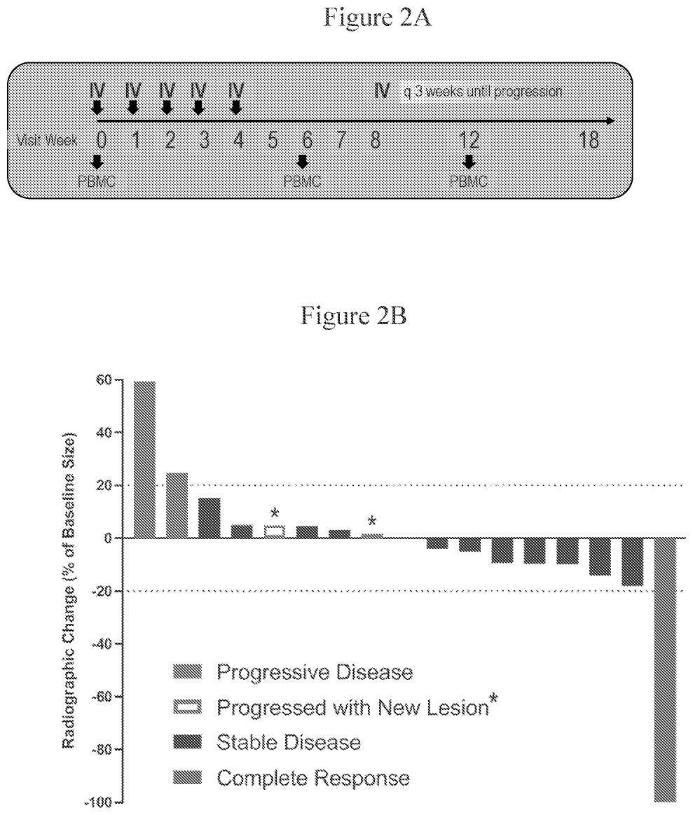

Patients were given Pexa-Vec (JX-594; a Wyeth vaccinia virus vaccine-derived oncolytic with disruption of the viral thymidine kinase gene and expression of the human granulocyte-monocyte colony stimulating factor (hGM-CSF) and β-galactosidase transgenes under control of the synthetic early-late and p7.5 promoters, respectively, see FIG. 1) intravenously at 10⁹ plaque-forming units (pfu) once a week for five weeks and every three weeks thereafter. Blood was collected for PBMC isolation at Baseline, Week 6, Week 12 and upon study completion. See FIG. 2A.

Stimulation of PBMCs with Peptides Corresponding to Renal Cell Carcinoma Antigens

Peripheral blood mononuclear cells (PBMC) were isolated from 16 renal cell carcinoma patients enrolled in the clinical trial at baseline (15/16 patients, prior to treatment) and at weeks 6 (16/16 patients) and 12 (12/16 patients) of a treatment protocol with the oncolytic vaccinia virus JX-594 (5 weekly intravenous infusions of 1×10⁹ pfu and every three weeks thereafter) and tested for immunoreactivity against a selection of 8 renal cell carcinoma-related antigens by ELISPOT: (i) RGS5=Regulator of G-protein signaling 5 (ii) MMP7=Matrix metallopeptidase 7 (iii) Survivin=BIRC5=baculoviral inhibitor of apoptosis repeat-containing 5 (iv) IGF-BP3=Insulin-like growth factor-binding protein 3 (v) MAGE-A3=Melanoma Antigen family A3 (Cancer/Testis Antigen Family 1, Member 3) (vi) TYMS=Thymidylate Synthetase (vii) HIG2=Hypoxia-inducible protein 2=Hypoxia Inducible Lipid Droplet Associated and (viii) PRUNE2=Prune Homolog 2. The 16 RCC patients were refractory to prior systemic treatments (mean (SD) of 2.9 lines of systemic therapy per patient including TKI/antioangiogenic, mTOR inhibitor, immunotherapeutic and/or chemotherapeutic treatments).

Seven of the eight antigens were used for stimulation as a peptide pool of 15mers overlapping by 11 amino acids spanning the entire protein. This format allows the efficient presentation of all potential class I- and class II-related epitopes to the patients' PBMC without the need to know patient HLA composition. In the case of PRUNE2, a single HLA-A2.01-restricted peptide was used for stimulation instead, which has been shown to elicit responses in RCC patients. The use of a peptide pool in this case was prohibitive due to the length of the protein (3088 amino acids=770 peptides).

A control peptide pool (CEFT) was included as an internal control for consistency of response status over time. The CEFT peptide pool consists of defined peptides from CMV, EBV, Influenza virus and Tetanus, which are known to elicit CD8 (CMV, EBV, Flu) and CD4 (Tetanus) responses in a consistent manner over time. For the assessment of background reactivity of each sample, PBMC were tested with medium plus DMSO alone (negative control). All samples were also assessed for overall functional status with non-specific anti-CD3 stimulation (positive control). Performance consistency (=Trending control) was assessed by simultaneous testing of the same External Reference PBMC sample against medium, CEFT and anti-CD3 in all experiments. Based on the trending control, the inter-assay variability (% CV) was determined to be <10.

PBMC from all time points of each patient were tested in the same experiment and an external reference sample included as a trending control between experiments. Each antigen condition was plated in triplicates, with 250,000 cells per well plated. The negative control (PBMC plus medium only) was plated in six replicates of 250,000 cells per well, to allow for proper statistical testing. As a positive control, 50,000 PBMC were stimulated with anti-CD3.

Elispot plates were read using a KS Elispot reader (Carl Zeiss Inc., Thornwood, N.Y.) equipped with KS Elispot Version 4.9.16 software. Spot parameters and evaluation algorithms were adapted to the specific occurrence of spots and background signals in each sample. All responses are based on Distribution-free Resampling (DFR)(eq) and DFR(2×) testing using the raw spot counts, none extrapolated. The cut-off for responses was 6 spots.

Results

Four patients (25%) were found to have developed responses against one or more RCC-related antigens after vaccination, which were not detectable at Baseline. Four patients (25%) exhibited one or more responses against RCC-related antigens already at Baseline, one of whom developed responses to other antigens after vaccination. All-together, responses against RCC-related antigens were detectable in 7 out of 16 patients (44%) at least one time point (BL, wk6. Wk12), including 4 patients with new responses after JX-594 treatment. Patients with T cell responses to the antigens all had stable disease, not progressive disease (i.e. all patients with T cell responses to the antigens were responders).

The response rate stratified by the clinical response (SD=stable disease; PD=progressive disease) at week 6 is shown at Table 1:

TABLE 1

| | | SD at 6 weeks | PD at 6 weeks |
|---|---|---|---|
| | | Response(DFR) | Response(DFR) |
| Antigen | Time Point | N (%) | N (%) |
| Survivin | Baseline | 0 (0%) | 0 (0%) |
| Survivin | Week 6 | 1 (8%) | 0 (0%) |
| Survivin | Week 12/ | 1 (9%) | 0 (0%) |
| | Complete | | |
| MAGEA3 | Baseline | 0 (0%) | 0 (0%) |
| MAGEA3 | Week 6 | 1 (8%) | 0 (0%) |
| MAGEA3 | Week 12/ | 0 (0%) | 0 (0%) |
| | Complete | | |
| PRUNE2 | Baseline | 0 (0%) | 0 (0%) |
| PRUNE2 | Week 6 | 1 (8%) | 0 (0%) |
| PRUNE2 | Week 12/ | 0 (0%) | 0 (0%) |
| | Complete | | |
| Vaccinia | Baseline | 1 (9%) | 2 (50%) |
| Vaccinia | Week 6 | 5 (42%) | 2 (50%) |
| Vaccinia | Week 12/ | 6 (55%) | 0 (0%) |
| | Complete | | |
| CEFT | Baseline | 9 (82%) | 2 (50%) |
| CEFT | Week 6 | 7 (58%) | 1 (25%) |
| CEFT | Week 12/ | 7 (64%) | 1 (100%) |
| | Complete | | |
| HIG2 | Baseline | 0 (0%) | 0 (0%) |
| HIG2 | Week 6 | 1 (8%) | 0 (0%) |
| HIG2 | Week 12/ | 1 (9%) | 0 (0%) |
| | Complete | | |
| TYMS | Baseline | 2 (18%) | 0 (0%) |
| TYMS | Week 6 | 2 (17%) | 0 (0%) |
| TYMS | Week 12/ | 2 (18%) | 0 (0%) |
| | Complete | | |
| RGS5 | Baseline | 2 (18%) | 0 (0%) |
| RGS5 | Week 6 | 2 (17%) | 0 (0%) |
| RGS5 | Week 12/ | 2 (18%) | 0 (0%) |
| | Complete | | |
| MMP7 | Baseline | 1 (9%) | 0 (0%) |
| MMP7 | Week 6 | 1 (8%) | 0 (0%) |
| MMP7 | Week 12/ | 1 (9%) | 0 (0%) |
| | Complete | | |
| IGF-BP3 | Baseline | 3 (27%) | 0 (0%) |
| IGF-BP3 | Week 6 | 4 (33%) | 0 (0%) |
| IGF-BP3 | Week 12/ | 3 (27%) | 0 (0%) |
| | Complete | | |

Response rate to ay tumor antigen are summarized at Table 2 (response rates by antigen and time point stratified by clinical response at 6 weeks):

TABLE 2

| | | SD at 6 weeks | PD at 6 weeks |
|---|---|---|---|
| | | Response(DFR) | Response(DFR) |
| Antigen | Time Point | N (%) | N (%) |
| Any Tumor* | Baseline | 4 (36%) | 0 (0%) |
| | Week 6 | 4 (33%) | 0 (0%) |
| | Week 12/Complete | 4 (36%) | 0 (0%) |
| Vaccinia | Baseline | 1 (9%) | 2 (50%) |
| | Week 6 | 5 (42%) | 2 (50%) |
| | Week 12/Complete | 6 (55%) | 0 (0%) |
| CEFT | Baseline | 9 (82%) | 2 (50%) |
| | Week 6 | 7 (58%) | 1 (25%) |
| | Week 12/Complete | 7 (64%) | 1 (100%) |

*A patient had a response to at least one of the 8 tumor antigens. At each time point there was a different combination of 4 patients that had a response to at least one antigen.

Six patients (38%) had a response to IGF-BP3 at least one of the three assessed time points (BL, wk6, wk12). Three out of the four patients who developed new responses against RCC-related antigens developed a response against IGF-BP3.

The results illustrate that a baseline (pretreatment) T lymphocyte response to certain renal cell carcinoma antigens, particularly IGF-BP3 (and to a lesser extent RGS5 and TYMS), in RCC patients is useful for identifying an RCC patient who will respond favorably to treatment with oncolytic vaccinia virus (exemplified here by JX-594).

### Example 2

A critical component to delineating anti-tumor activity of oncolytic viruses is to monitor peripheral immune response to the virus. Assays to monitor functional CD8+T cell responses in the blood patients treated with oncolytic vaccinia virus JX-594 were developed.

Peripheral blood mononuclear cells (PBMC) from 17 patients enrolled in the clinical trial described at Example 1 were collected by Ficoll separation and cryopreserved prior to treatment, at six and twelve weeks post-Pexa-Vec initiation, and upon completion of study. Clinical response was determined for all patients according to mRECIST1.0 criteria at weeks six, twelve, and upon study completion: Stable Disease (<20% increase and <20% decrease in lesion size), Progressive Disease (>20% increase), or Response (>20% decrease). See FIG. 2B.

This example compares the use of an overlapping vaccina peptide pool (covering the second half of Major core protein P4a of vaccinia virus) to the use of epitope-based peptide pools to track peripheral CD8+T cell response to vaccinia virus in patients enrolled in the clinical trial.

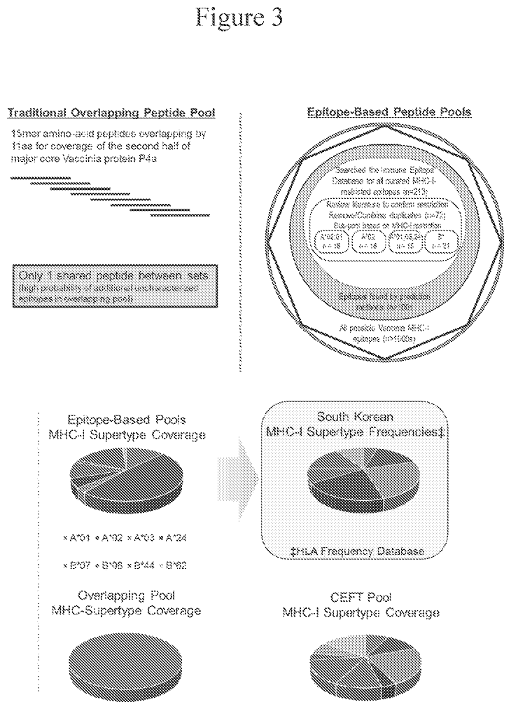

As described in detail below, the custom peptide pools were designed from known immunogenic vaccinia virus epitopes in an HLA-agnostic format to profile peripheral CD8+T cell responses. All curated MHC class I-restricted epitopes to vaccinia virus were retrieved from the Immune Epitope Database and duplicates were removed. The remaining epitopes (n=70) were synthesized and pooled based on known HLA restriction. See FIG. 3. Individual pools comprised no more than 20 peptides. PBMCs were stimulated for 48 hours and response to peptide pools were measured by dual IFNγ/granzyme B ELISPOT. Additionally, patient HLA haplotypes were determined by sequencing and all samples were evaluated by flow cytometry to assess T cell phenotypes.

Overlapping Vaccinia Peptide Pool ELISPOT Testing

Briefly, patient PBMCs were assayed by IFNγELISPOT (R&D Systems, Minneapolis, Minn.) for response to a Vaccinia peptide pool containing 104 peptides of 15 residues in length overlapping by 10 covering the second half of Major core protein P4a of vaccinia virus (JPT Peptide Technologies, Acton, Mass.).

Vaccinia Epitope-Based Peptide Pool Design

All peptides included in the study had been identified as human HLA class I epitopes for Vaccinia and curated by the Immune Epitope Database. Search criteria for peptides were as follows: Linear Epitope; Vaccinia virus (ID:10245); Positive Assays Only; T Cell Assays; MHC Class I; Humans; Any Disease; Any Reference Type. From the resulting 212 epitopes, specific HLA class I restriction was noted, and only epitopes with positive reactivity in at least two human donors confirmed through a literature search were included. Additionally, overlapping epitope sequences with identical HLA class I restriction were combined provided the resulting sequence was under eleven residues in length. The 70 remaining epitopes (Table 3) were synthesized at Genscript (Piscataway, N.J.) to >90% purity, reconstituted in DMSO, and pooled based on reported HLA class I restriction as noted on Table 3. Individual pools were comprised of no more than 20 peptides.

TABLE 3

| | HLA class I | | | | |
|---|---|---|---|---|---|
| Pool | Restriction | Parent Protein | Sequence | Start | End |
| A*02 | A*02 | DNA polymerase | FLNISWFYI (SEQ ID | 107 | 115 |
| supertype | | | NO: 1) | | |
| A*02 | A*02 | Early transcription factor 82 kDa | FLVIAINAM (SEQ ID | 342 | 350 |
| supertype | | subunit | NO: 2) | | |
| A*02 | A*02 | Intermediate transcription factor 3 | YLFRCVDAV (SEQ ID | 72 | 80 |
| supertype | | small subunit | NO: 3) | | |
| A*02 | A*02 | Kelch repeat and BTB domain- | YIYGIPLSL (SEQ ID | 78 | 86 |
| supertype | | containing protein A55 | NO: 4) | | |
| A*02 | A*02 | mRNA-capping enzyme regulatory | SLFKNVRLL (SEQ ID | 174 | 182 |
| supertype | | subunit | NO: 5) | | |
| A*02 | A*02 | Plaque-size/host range protein | SVVTLLCVLPAVVYS | 5 | 19 |
| supertype | | | (SEQ ID NO: 6) | | |
| A*02 | A*02 | Poly(A) polymerase catalytic | FLIDLAFLI (SEQ ID | 213 | 221 |
| supertype | | subunit | NO: 7) | | |
| A*02 | A*02 | Profilin | LMDENTYAM (SEQ | 74 | 82 |
| supertype | | | ID NO: 8) | | |
| A*02 | A*02 | Protein A36 | MMLVPLITV(SEQ ID | 1 | 9 |
| supertype | | | NO: 9) | | |
| A*02 | A*02 | Protein A6 | ILSDENYLL (SEQ ID | 172 | 180 |
| supertype | | | NO: 10) | | |
| A*02 | A*02 | Protein E2 | KIDYYIPYV (SEQ ID | 249 | 257 |
| supertype | | | NO: 11) | | |
| A*02 | A*02 | Protein F12 | NLFDIPLLTV(SEQ ID | 286 | 295 |
| supertype | | | NO: 12) | | |
| A*02 | A*02 | Protein F12 | FLTSVINRV (SEQ ID | 404 | 412 |
| supertype | | | NO: 13) | | |
| A*02 | A*02 | Protein N1 | RMIAISAKV(SEQ ID | 71 | 79 |
| supertype | | | NO: 14) | | |
| A*02 | A*02 | Protein N2 | YVNAILYQI (SEQ ID | 93 | 101 |
| supertype | | | NO: 15) | | |
| A*02 | A*02 | Protein O1 | GLNDYLHSV (SEQ ID | 247 | 255 |
| supertype | | | NO: 16) | | |
| A*02 | A*02 | Ribonucleoside-diphosphate | SMHFYGWSL (SEQ | 720 | 728 |
| supertype | | reductase large subunit | ID NO: 17) | | |
| A*02 | A*02 | RNA helicase NPH-II | KLLLWFNYL (SEQ ID | 197 | 205 |
| supertype | | | NO: 18) | | |
| A*02: 01 pool | A*02: 01 | A-type inclusion protein A25 | YLYTEYFLFL (SEQ ID | 177 | 186 |
| | | | NO: 19) | | |
| A*02: 01 pool | A*02: 01 | DNA-directed RNA polymerase 7 | SLKDVLVSV (SEQ ID | 27 | 35 |
| | | kDa subunit | NO: 20) | | |
| A*02: 01 pool | A*02: 01 | Envelope protein H3 | SLSAYIIRV (SEQ ID | 184 | 192 |
| | | | NO: 21) | | |
| A*02: 01 pool | A*02: 01 | Interferon antagonist C7 | KVDDTFYYV (SEQ ID | 74 | 82 |
| | | | NO: 22) | | |
| A*02: 01 pool | A*02: 01 | Intermediate transcription factor 3 | ALDEKLFLI (SEQ ID | 273 | 281 |
| | | large subunit | NO: 23) | | |
| A*02: 01 pool | A*02: 01 | Kelch repeat and BTB domain- | AMLNGLIYV (SEQ ID | 391 | 399 |
| | | containing protein A55 | NO: 24) | | |
| A*02: 01 pool | A*02: 01 | mRNA-capping enzyme regulatory | KLFTHDIML (SEQ ID | 62 | 70 |
| | | subunit | NO: 25) | | |
| A*02: 01 pool | A*02: 01 | mRNA-capping enzyme regulatory | RVYEALYYV (SEQ ID | 251 | 259 |
| | | subunit | NO: 26) | | |
| A*02: 01 pool | A*02: 01 | Protein A47 | LLYAHINAL (SEQ ID | 155 | 163 |
| | | | NO: 27) | | |
| A*02: 01 pool | A*02: 01 | Protein B14 | CLTEYILWV (SEQ ID | 79 | 87 |
| | | | NO: 28) | | |
| A*02: 01 pool | A*02: 01 | Protein B14 | TLLDHIRTA (SEQ ID | 137 | 145 |
| | | | NO: 29) | | |
| A*02: 01 pool | A*02: 01 | Protein E5 | KLFSDISAI (SEQ ID | 107 | 115 |
| | | | NO: 30) | | |
| A*02: 01 pool | A*02: 01 | Putative nuclease G5 | ILDDNLYKV (SEQ ID | 18 | 26 |
| | | | NO: 31) | | |
| A*02: 01 pool | A*02: 01 | Soluble interferon alpha/beta | KLIIHNPEL (SEQ ID | 207 | 215 |
| | | receptor B18 | NO: 32) | | |
| A*02: 01 pool | A*02: 01 | Soluble interferon gamma receptor | KITSYKFESV (SEQ ID | 18 | 27 |
| | | B8 | NO: 33) | | |
| A*02: 01 pool | A*02: 01 | Thymidylate kinase | IVIEAIHTV (SEQ ID | 187 | 195 |
| | | | NO: 34) | | |
| Other A* pool | A*01 | mRNA-capping enzyme regulatory | GTHVLLPFY (SEQ ID | 11 | 19 |
| | | subunit | NO: 35) | | |
| Other A* pool | A*01 | Protein A27 | LRAAMISLAKKIDVQ | 89 | 103 |
| | | | (SEQ ID NO: 36) | | |
| Other A* pool | A*01 | Protein A38 | VSEHFSLLF (SEQ ID | 257 | 265 |
| | | | NO: 37) | | |
| Other A* pool | A*01 | Protein C10 | QSDTVFDYY(SEQ ID | 298 | 306 |
| | | | NO: 38) | | |
| Other A* pool | A*01 | Soluble interferon gamma receptor | DMCDIYLLY(SEQ ID | 139 | 147 |
| | | B8 | NO: 39) | | |
| Other A* pool | A*01 | Soluble interferon gamma receptor | FGDSKEPVPY (SEQ | 153 | 162 |
| | | B8 | ID NO: 40) | | |
| Other A* pool | A*01 | Transcript termination protein A18 | LSDLKKTIY (SEQ ID | 255 | 263 |
| | | | NO: 41) | | |
| Other A* pool | A*01: 01 | DNA-directed RNA polymerase 133 | ITDFNIDTY(SEQ ID | 278 | 286 |
| | | kDa polypeptide | NO: 42) | | |
| Other A* pool | A*03 | Intermediate transcription factor 3 | AVKDVTITKK (SEQ | 79 | 88 |
| | | small subunit | ID NO: 43) | | |
| Other A* pool | A*03 | Protein C5 | KVMFVIRFK(SEQ ID | 158 | 166 |
| | | | NO: 44) | | |
| Other A* pool | A*23: 01 | Thymidylate kinase | TYNDHIVNL (SEQ ID | 58 | 66 |
| | | | NO: 45) | | |
| Other A* pool | A*23: 01 | Primase D5 | VWINNSWKF (SEQ | 349 | 357 |
| | | | ID NO: 46) | | |
| Other A* pool | A*24 | DNA polymerase processivity factor | KYQSPVNIF (SEQ ID | 144 | 152 |
| | | component A20 | NO: 47) | | |
| Other A* pool | A*26 | Major core protein 4a precursor | DTRGIFSAY (SEQ ID | 636 | 644 |
| | | | NO: 48) | | |
| Other A* pool | A*29: 03/02 | Serine proteinase inhibitor 1 | VYINHPFMY (SEQ ID | 326 | 334 |
| | | | NO: 49) | | |
| B* pool | B*07 | Protein B14 | TVADVRHCL (SEQ ID | 53 | 61 |
| | | | NO: 50) | | |
| B* pool | B*07: 02 | DNA-directed RNA polymerase 147 | MPAYIRNTL (SEQ ID | 303 | 311 |
| | | kDa polypeptide | NO: 51) | | |
| B* pool | B*07: 02 | mRNA-capping enzyme catalytic | HPRHYATVM (SEQ | 686 | 694 |
| | | subunit | ID NO: 52) | | |
| B* pool | B*07: 02 | Protein C1 | KPKPAVRFAI (SEQ | 102 | 111 |
| | | | ID NO: 53) | | |
| B* pool | B*07: 02 | Protein O1 | RPMSLRSTII (SEQ ID | 335 | 344 |
| | | | NO: 54) | | |
| B* pool | B*07: 02 | Ribonucleoside-diphosphate | APNPNRFVI (SEQ ID | 6 | 14 |
| | | reductase small chain | NO: 55) | | |
| B* pool | B*08: 01 | DNA ligase | WLKIKRDYL (SEQ ID | 395 | 403 |
| | | | NO: 56) | | |
| B* pool | B*15: 01 | Protein K7 | SIIDLIDEY (SEQ ID | 25 | 33 |
| | | | NO: 57) | | |
| B* pool | B*35 | Plaque-size/host range protein | CIDGKWNPILPTCVR | 225 | 239 |
| | | | (SEQ ID NO: 58) | | |
| B* pool | B*44 | Dual specificity protein | SEVKFKYVL (SEQ ID | 48 | 56 |
| | | phosphatase H1 | NO: 59) | | |
| B* pool | B*44 | Plaque-size/host range protein | TKYFRCEEKNGNTSW | 105 | 119 |
| | | | (SEQ ID NO: 60) | | |
| B* pool | B*44: 03 | lnterleukin-18-binding protein | DEIKCPNLN (SEQ ID | 21 | 29 |
| | | | NO: 61) | | |
| B* pool | B*44: 03 | Intermediate transcription factor 3 | HDVYGVSNF (SEQ | 287 | 295 |
| | | large subunit | ID NO: 62) | | |
| B* pool | B*44: 03 | Major core protein 4b | DEVASTHDW (SEQ | 90 | 98 |
| | | | ID NO: 63) | | |
| B* pool | B*44: 03 | Major core protein 4b | YEFRKVKSY (SEQ ID | 264 | 272 |
| | | | NO: 64) | | |
| B* pool | B*44: 03 | mRNA-capping enzyme catalytic | EERHIFLDY (SEQ ID | 126 | 134 |
| | | subunit | NO: 65) | | |
| B* pool | B*44: 03 | Primase D5 | LENGAIRIY (SEQ ID | 298 | 306 |
| | | | NO: 66) | | |
| B* pool | B*44: 03 | Primase D5 | EEIPDFAFY (SEQ ID | 691 | 699 |
| | | | NO: 67) | | |
| B* pool | B*44: 03 | Protein E3 | DDVSREKSM (SEQ | 86 | 94 |
| | | | ID NO: 68) | | |
| B* pool | B*44: 03 | Protein I3 | IEGELESLS (SEQ ID | 173 | 181 |
| | | | NO: 69) | | |
| B* pool | B*44: 03 | Protein M2 | AELTIGVNY (SEQ ID | 38 | 46 |
| | | | NO: 70) | | |

ELISPOT Assay

All samples were assayed for their response to the Vaccinia overlapping peptide pool or to the novel epitope-based peptide pools by ELISPOT as follows. Cryopreserved PBMCs were thawed and allowed to rest for 16 hours in media with human serum at 37° C. Subsequently, the PBMCs were harvested and stimulated at 200,000 cells per well with the peptides pools at 5 μg/ml per individual peptide in 200 ul using the Human IFNγ/GranzymeB Double-Color FluoroSpot (ImmunoSpot, Cleveland, Ohio). Stimulations were incubated for 48 hours at 37° C. and developed using the manufacturer's protocol. Fluorescent spot forming cells (SFC) were imaged and counted on an ImmunoSpot S6 Universal. Subsequent analyses of the data were done on Microsoft Excel and Graphpad Prism.

Overlapping Peptide Pool Results

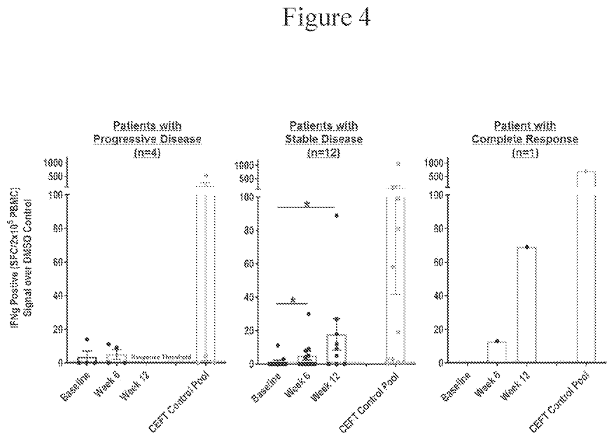

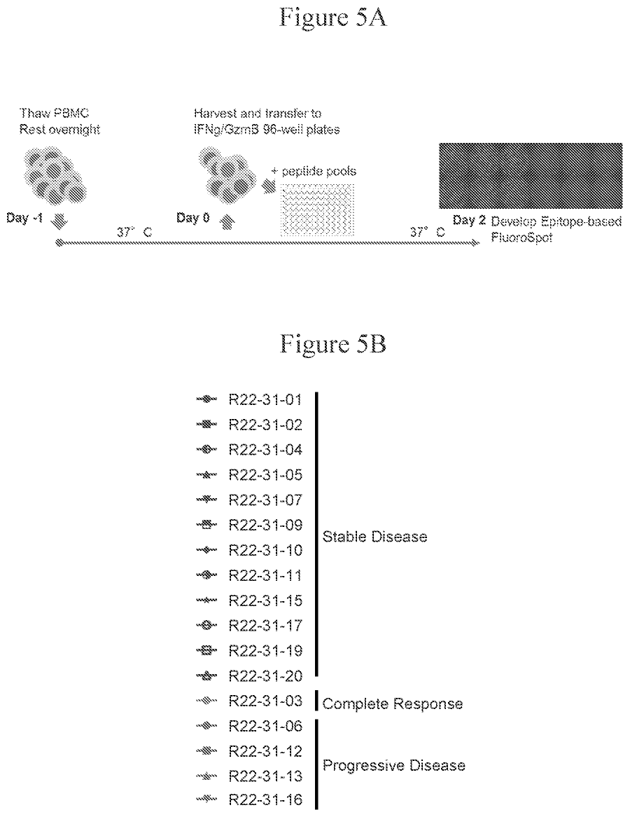

Initially, anti-Vaccinia T cell responses were determined to an overlapping peptide pool covering the second half of the Vaccinia protein P4a. PBMCs from patients were stimulated with the peptide pool for 24 hours and responding cells were quantified by IFNγELISPOT. Of note, for the majority of samples, the ELISPOT assay had a particularly high background (30-60 SFC) and/or a large variability between the triplicate wells for each stimulation. To normalize analysis, the background spot detection was subtracted from each sample. Due to the high variation of replicates, only responses greater than 10 SFC were considered above the level of detection. The results are illustrated at FIG. 4.

For the majority of samples, the assay with the overlapping peptide pool had a particularly high background (30-60 SFC) and/or a large variability between the triplicate wells for each stimulation. Interestingly, in patients with Stable Disease, an increase in the response to the overlapping peptide pool was observed (*p<0.05, paired non-parametric Student's t-test). Previous studies have found strong peripheral T cell responses to Vaccinia for many years after vaccination, so it was likely that the chosen overlapping pool did not contain sufficient epitope specific to accurately measure the response. Unfortunately, a full screen of all potential Vaccinia epitopes would consist of thousands of peptides and require larger amounts of PBMC from patients than what could be safely collected. Thus, we concluded that this format of the assay is insufficient to accurately track peripheral responses to Pexa-Vec.

Epitope-Based Peptide Pool Results

Increased Detection of Responses to Vaccinia Virus

As the detected response to the overlapping vaccinia peptide pool was below what was expected, custom peptide pools from known immunogenic Vaccinia epitopes in an HLA-agnostic format to profile peripheral CD8⁺T cell responses as described above in order to accurately track anti-Vaccinia responses in these patients and efficiently use small volumes of collected blood.

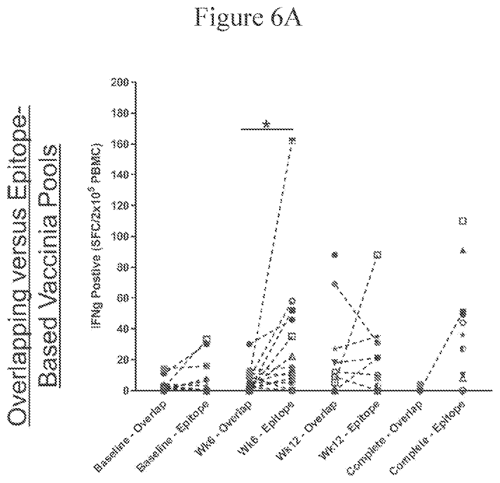

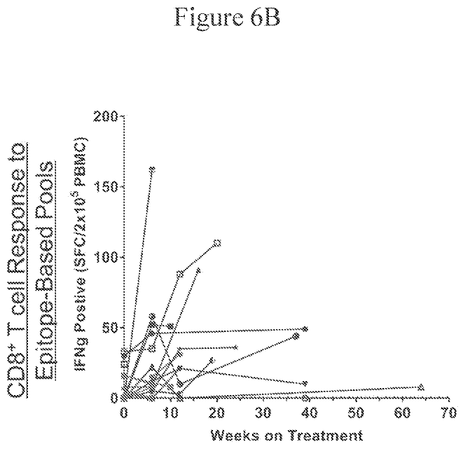

Upon screening PBMC from patients included in the study with the new peptide pools in the 48-hour ELISPOT as described above, we found that the epitope-based, HLA-agnostic peptide pools were successful in tracking CD8⁺T cell responses in RCC patients treated with the oncolytic virus Pexa-Vec, with increased response detected for almost every sample with epitope-based pools in comparison to the overlapping pool. See FIG. 6A. At Baseline and Week 6, this difference between the two stimuli was significantly different (*p<0.05, paired non-parametric Student's t-test) and the trend was still evident at Baseline and Week 12. Additionally, the responses to each of the two pool formats were compared using specific thresholds of detection. Using response thresholds of 10 SFC (p<0.05) or 20 SFC (p<0.005) per 2×10⁵ PBMC, the anti-vaccinia response to the epitope-based pools were statistically higher than the overlapping peptide pool, with there being statistically more patients with responses higher than 10 SFC (p<0.05) and 20 SFC (p<0.005) with the epitope-based pools (Table 4, see also FIG. 6B):

TABLE 4

| | >10 | <10 | >20 | <20 | |
|---|---|---|---|---|---|
| | SFC | SFC | SFC | SFC | Fisher’s exact test |
| Overlapping | 6 | 11 | 3 | 14 | <0.05 |
| Epitope-Based | 13 | 4 | 12 | 5 | <0.005 |

Overall, these data show that the epitope-based pools are a more accurate method to detect peripheral responses to vaccinia in these patients than the overlapping peptide pool.

Response to Epitope-Based Peptide Pools at Week 6 Correlates with Clinical Response

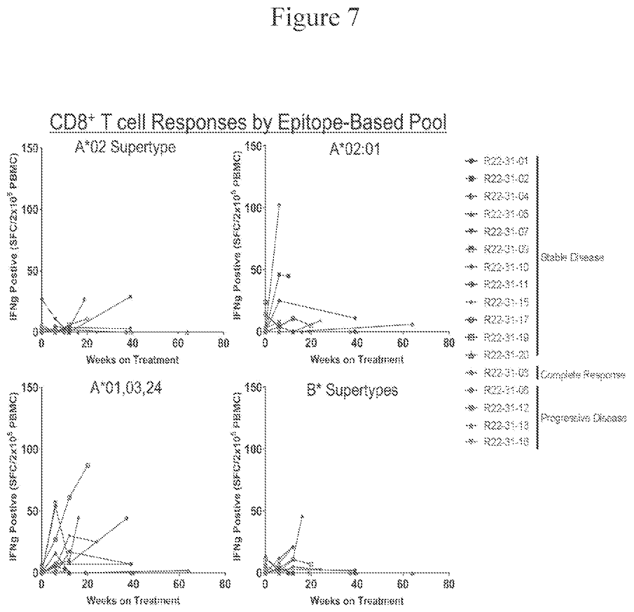

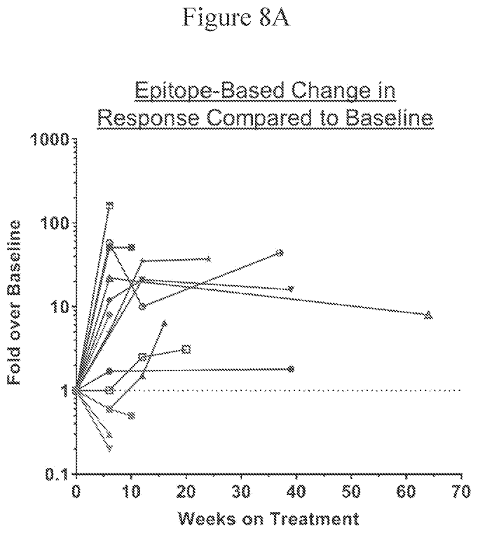

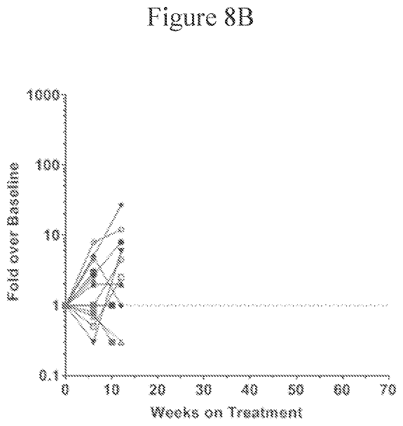

Consistent with the HLA allele frequencies of the South Korean population, most of the induction was specific to the A*01,03,24 pool, but there was also a large amount of induction in the A*02:01 pool (see FIG. 7). To determine whether there is a correlation between induction of T-cell response and clinical response, the SFC measured on treatment was normalized to the response at Baseline for each patient. To calculate the “fold change” of each patient longitudinally as compared to the Baseline response, the absolute SFC value at Week 6, Week 12, or additional time-points were divided by the absolute value of the Baseline response. Thus, a fold change of greater than 1 is an increase in response, and less than 1 is a decrease. See FIG. 8A. Interestingly, the majority (8/11) of patients with Stable Disease had a measurable increase in T cell response to epitope-based pools at Week 6 and beyond. Additionally, no Stable Disease patients displayed a decreased overall response. In contrast, three of four patients with Progressive Disease decreased their response to the pools at Week 6 (p=0.0517; Table 5):

TABLE 5

| | Increased | No or | |
|---|---|---|---|
| | Response | Decreased | |
| | post vaccination | Response | Fisher’s exact test |
| Stable Disease | 8 | 1 | 0.0517 |
| Progressive Disease | 1 | 3 | |

Further, the same analysis performed on the response to the overlapping pool did not show the same grouping of inductions as the epitope-based pools. Rather, the change in response did not trend with any specific clinical response. See FIG. 8B.

Correlations with Clinical and Peripheral Epitope-Based Responses

Finally, we investigated the correlations between the fold change in peripheral T cell response to clinical observations. The change in response at week 6 for all patients was analyzed in comparison to clinical response, excluding patients for which a baseline or week 6 PBMC sample was not available to assay or for which no response to the vaccinia pools were detected. Correlation was determined by linear regression of all patients.

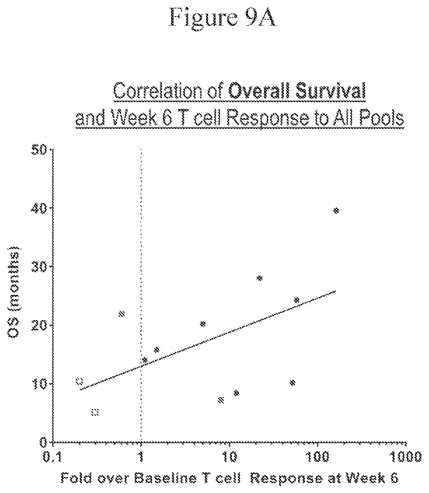

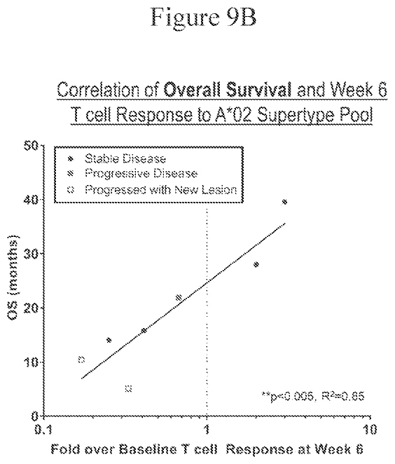

A trend of correlation was observed between overall survival and the combined response to all of the vaccinia epitope-based pools. See FIG. 9A. A strong correlation between overall survival and the response to the A*02 supertype pool was observed. See FIG. 9B.

### CONCLUSIONS

The results demonstrate that the HLA-agnostic peptide pools were successful in tracking CD8+T cell responses in RCC patients treated with JX-594. Interestingly, 8/11 patients with Stable Disease at week six had increased responses to the peptide pools relative to baseline, whereas three out of four Progressive Disease patients displayed decreased responses (p<0.005, Fisher t test). Additionally, an increase in PBMC response at week six to the A*02 supertype pool directly correlated to overall survival of the patients, whereas no correlation was detected with the other peptide pools.

Thus, profiling of the peripheral CD8+T cell responses to vaccinia virus (exemplified here by JX-594) can be achieved with a high degree of accuracy and minimal sample requirements in an HLA-agnostic manner using custom peptide pools based on known Vaccinia epitopes. This method allows for screening with small peptide pools numbers (10s rather than 100-1000s) and thus requires significantly fewer valuable clinical trial samples. By reducing and improving the number of candidate epitopes, the assay can track systemic patient responses while using less of the valuable clinical samples.

The CD8⁺T cell response to Pexa-Vec at Week 6 after treatment initiation suggests that patients with Stable disease had a higher induction of T cell response to epitope-based Vaccinia peptide pools than those with Progressive disease.

The peptide pools can be used to predict clinical responses to oncolytic vaccinia virus at early time points in a variety of cancers. Paired tumor biopsies allow the correlation with vaccinia-specific TCRs identified in the periphery with TCRs on intra-tumoral CD8+T cells.

### Example 3

The epitope-based peptide pools described above in Example 2 were used to evaluate the circulating vaccinia virus-specific CD8+T cell response in patients with advanced renal cell carcinoma (naive or refractory to prior systemic treatment and who had no prior treatment with immune checkpoint inhibitors) during the course of treatment with 4 weekly intravenous infusions of JX-594 (Pexa-Vec) at 10⁹ plaque forming units (pfu) starting at Day-7 plus the monoclonal anti-PD-1 antibody Cemiplimab (350 mg every 3 weeks) from Day 1. Radiographic assessments per RECIST 1.1 were performed centrally every 9 weeks from Day 1. Peripheral blood mononuclear cells (PBMCs) were collected and cryopreserved at baseline and at 29 days post initial JX-594 treatment.

IFNγELISPOT analysis was performed on longitudinal PMBC samples using the epitope-based peptide pools described above in Example 2 (OV peptides) and culture conditions designed to measure existing oncolytic virus (OV)-specific memory T cell cytolytic activity. PBMC samples from patients were tested for IFNγ release following stimulation with OV peptides using two different assay conditions: (1) measurement following direct ex vivo stimulation with OV peptides alone and (2) measurement following 10 days of T cell expansion in the presence of OV peptides, the T cell supportive cytokines GM-CSF, IL-4, IL-7 and IL-15 and autologous dendritic cells. The number of OV-specific IFNγ spots was correlated with the clinical response and tumor regression.

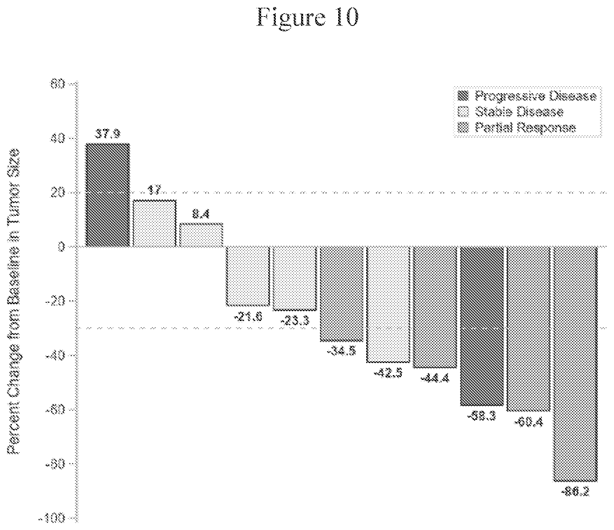

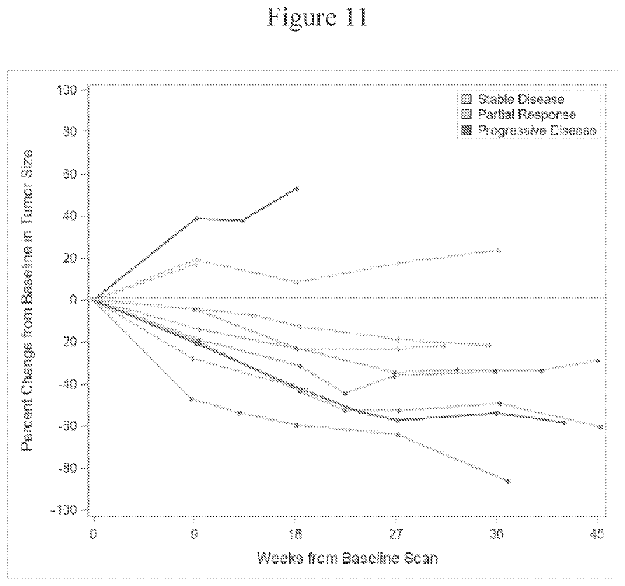

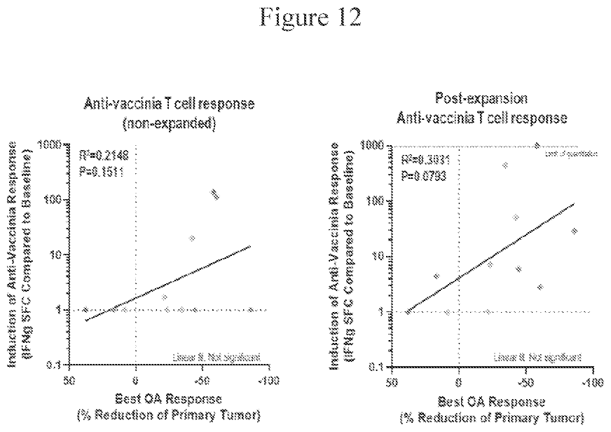

Remarkably, 8 of the 11 (72.7%) patients showed tumor burden reduction, 4 of whom had ≥30% confirmed reduction (FIGS. 10 and 11). OV-specific IFNγ-producing T cells were detected in only 3 of 11 patients in the nonexpanded ELISPOT culture conditions (FIG. 12, left panel), but in 8 out of 11 patients when T cells were first expanded for 10 days in the presence of OV peptides prior to ELISPOT, which trended toward a correlation with the preliminary clinical response assessment (FIG. 12, right panel). Prolonged stimulation with CMV, EBV and Influenza peptides did not show any correlation (R²=0.005), suggesting that the treatment and culture expansion influenced relevant OV-specific memory T cell proliferation. There was a 9-fold increase in the detection range of IFNγ producing cells up to 1000 SFC/2×10⁵ cells (FIG. 12, right panel), from a detection range of fewer than 110 SFC/2×10⁵ cells (FIG. 12, left panel). Pre-expanded ELISPOT revealed a trend of correlation between CD8+T cell response and tumor volume reduction even at an early sample collection time point (Day 29) with R2=0.3031 and p-value=0.0793.

Conclusion: These results demonstrate that OV-specific T cell responses can be induced by OV therapy. In addition, 10-day expansion of low levels of OV-specific circulating T cells can amplify signals in ELISPOT analysis and might enable systemic tracking of patient responses in blood samples collected at early time points. The observed CD8+T cell response to oncolytic vaccinia virus in patients supports the rationale for combination treatment with OV (e.g. Pexa-Vec) and immune checkpoint inhibitors (e.g. Cemiplimab).

While the materials and methods of this invention have been described in terms of preferred embodiments, it will be apparent to those of skill in the art that variations may be applied to the method described herein without departing from the concept, spirit and scope of the invention. All such similar substitutes and modifications apparent to those skilled in the art are deemed to be within the spirit, scope and concept of the invention

1. A composition for use in measuring an immune response against an oncolytic vaccinia virus, the composition comprising a population of peptides comprising at least 5 different peptides, wherein each peptide in the population consists essentially of an amino acid sequence selected from those set forth in SEQ ID NOs:1-70.

2. A composition for use according to claim 1, comprising a population of peptides, each peptide consisting essentially of an amino acid sequence forth in SEQ ID NOs: 1-18.

3. A composition for use according to claim 1, comprising a population of peptides, each peptide consisting essentially an amino acid sequence forth in SEQ ID NOs: 19-34.

4. A composition for use according to claim 1, comprising a population of peptides, each peptide consisting essentially of an amino acid sequence forth in SEQ ID NOs: 35-49.

5. A composition for use according to claim 1, comprising a population of peptides, each peptide consisting essentially of an amino acid sequence forth in SEQ ID NOs: 50-70.

6. A method for monitoring the number and/or status of vaccinia virus-reactive T cells in a patient that has received one or more oncolytic vaccinia virus treatments, the method comprising determining the patient's immune reactivity to a composition according to claim 1.

7. A method for determining the prognosis of a cancer patient that has received one or more oncolytic vaccinia virus treatments, the method comprising determining the patient's immune reactivity to a composition according to claim 1.

8. A method for monitoring the efficacy of vaccinia virus treatment in a cancer patient that has been administered one or more doses of oncolytic vaccinia virus, comprising determining the patient's immune reactivity to a composition according to claim 1.

9. A method for monitoring the efficacy of a combination therapy comprising co-administration of an oncolytic vaccinia virus and one or more checkpoint inhibitors in a cancer patient that been administered one or more doses of oncolytic vaccinia virus and one or more doses of a checkpoint inhibitor, comprising determining the patient's immune reactivity to a composition according to claim 1.

10. The method of claim 6, comprising contacting a PBMC or whole blood sample from the patient with one or more compositions according to any one of claims 2-5.

11. The method of claim 6, wherein the patient's immune reactivity to the one or more compositions is assessed by ELISPOT assay.

12. The method of claim 11, wherein the ELISPOT assay quantifies the level of IFN-γ produced by CD8+T cells in response to the one or more compositions.

13. The method of claim 6, wherein the sample obtained from the patient is incubated with the one or more compositions for a period of at least 12, at least 24, at least 36 or at least 48 hours.

14. The method of claim 6, wherein T cells in the sample are expanded ex vivo in the presence of(i) the one or more compositions (ii) autologous antigen presenting cells, preferably dendritic cells, and (iii) one or more T cell supportive cytokines.

15. The method of claim 14, wherein unfractionated PBMCs are directly stimulated in culture with the one or more compositions, IL-4, IL-7, IL-15 and GM-CSF and wherein the culture does not comprise isolated antigen presenting cells pre-stimulated with the one or more compositions.

16. The method according to claim 6, wherein the patient has a cancer selected from brain cancer, renal cancer, liver cancer, lung cancer, head and neck cancer, breast cancer, pancreatic cancer, prostate cancer, bone cancer, testicular cancer, cervical cancer, ovarian cancer, uterine cancer, rectal cancer, gastrointestinal cancer, lymphoma, pre-neoplastic lesions in the lung, colon cancer, melanoma, and bladder cancer.

17. The method according to claim 16, wherein the patient has a cancer selected from renal cell carcinoma, hepatocellular carcinoma and melanoma.

18. The method according to claim 6, wherein the patient received one or more treatments of a wild type or genetically modified Wyeth or Western Reserve strain vaccinia virus.

19. The method according to claim 18, wherein the patient received one or more treatments of a genetically modified Wyeth or Western Reserve strain vaccinia virus lacking a functional thymidine kinase gene and/or lacking a functional vaccinia growth factor gene.

20. (canceled)

21. The method according to claim 19, wherein the Wyeth or Western Reserve strain vaccinia virus contained a transgene encoding a cytokine, preferably a transgene encoding GM-CSF.

22. The method according to claim 22, wherein the patient received one or more intratumoral and/or intravenous treatments of JX-594.

23. (canceled)

24. The method according to claim 6, wherein the vaccinia virus was administered to the patient at a dose of 10⁸ to 10⁹ pfu.

25. The method according to claim 6, wherein the patient was co-administered one or more immune checkpoint inhibitors.

26. (canceled)


SEQUENCE LISTING (70 sequences)

SEQ ID NO: 1 - protein, 9 aa, Artificial Sequence
  human HLA class I Vaccinia epitope
FLNISWFYI

SEQ ID NO: 2 - protein, 9 aa, Artificial Sequence
  human HLA class I Vaccinia epitope
FLVIAINAM

SEQ ID NO: 3 - protein, 9 aa, Artificial Sequence
  Human HLA class I Vaccinia epitope
YLFRCVDAV

SEQ ID NO: 4 - protein, 9 aa, Artificial Sequence
  Human HLA class I Vaccinia epitope
YIYGIPLSL

SEQ ID NO: 5 - protein, 9 aa, Artificial Sequence
  Human HLA class I Vaccinia epitope
SLFKNVRLL

SEQ ID NO: 6 - protein, 15 aa, Artificial Sequence
  Human HLA class I Vaccinia epitope
SVVTLLCVLPAVVYS

SEQ ID NO: 7 - protein, 9 aa, Artificial Sequence
  Human HLA class I Vaccinia epitope
FLIDLAFLI

SEQ ID NO: 8 - protein, 9 aa, Artificial Sequence
  Human HLA class I Vaccinia epitope
LMDENTYAM

SEQ ID NO: 9 - protein, 9 aa, Artificial Sequence
  Human HLA class I Vaccinia epitope
MMLVPLITV

SEQ ID NO: 10 - protein, 9 aa, Artificial Sequence
  Human HLA class I Vaccinia epitope
ILSDENYLL

SEQ ID NO: 11 - protein, 9 aa, Artificial Sequence
  Human HLA class I Vaccinia epitope
KIDYYIPYV

SEQ ID NO: 12 - protein, 10 aa, Artificial Sequence
  Human HLA class I Vaccinia epitope
NLFDIPLLTV

SEQ ID NO: 13 - protein, 9 aa, Artificial Sequence
  Human HLA class I Vaccinia epitope
FLTSVINRV

SEQ ID NO: 14 - protein, 9 aa, Artificial Sequence
  Human HLA class I Vaccinia epitope
RMIAISAKV

SEQ ID NO: 15 - protein, 9 aa, Artificial Sequence
  Human HLA class I Vaccinia epitope
YVNAILYQI

SEQ ID NO: 16 - protein, 9 aa, Artificial Sequence
  Human HLA class I Vaccinia epitope
GLNDYLHSV

SEQ ID NO: 17 - protein, 9 aa, Artificial Sequence
  Human HLA class I Vaccinia epitope
SMHFYGWSL

SEQ ID NO: 18 - protein, 9 aa, Artificial Sequence
  Human HLA class I Vaccinia epitope
KLLLWFNYL

SEQ ID NO: 19 - protein, 10 aa, Artificial Sequence
  Human HLA class I Vaccinia epitope
YLYTEYFLFL

SEQ ID NO: 20 - protein, 9 aa, Artificial Sequence
  Human HLA class I Vaccinia epitope
SLKDVLVSV

SEQ ID NO: 21 - protein, 9 aa, Artificial Sequence
  Human HLA class I Vaccinia epitope
SLSAYIIRV

SEQ ID NO: 22 - protein, 9 aa, Artificial Sequence
  Human HLA class I Vaccinia epitope
KVDDTFYYV

SEQ ID NO: 23 - protein, 9 aa, Artificial Sequence
  Human HLA class I Vaccinia epitope
ALDEKLFLI

SEQ ID NO: 24 - protein, 9 aa, Artificial Sequence
  Human HLA class I Vaccinia epitope
AMLNGLIYV

SEQ ID NO: 25 - protein, 9 aa, Artificial Sequence
  Human HLA class I Vaccinia epitope
KLFTHDIML

SEQ ID NO: 26 - protein, 9 aa, Artificial Sequence
  Human HLA class I Vaccinia epitope
RVYEALYYV

SEQ ID NO: 27 - protein, 9 aa, Artificial Sequence
  Human HLA class I Vaccinia epitope
LLYAHINAL

SEQ ID NO: 28 - protein, 9 aa, Artificial Sequence
  Human HLA class I Vaccinia epitope
CLTEYILWV

SEQ ID NO: 29 - protein, 9 aa, Artificial Sequence
  Human HLA class I Vaccinia epitope
TLLDHIRTA

SEQ ID NO: 30 - protein, 9 aa, Artificial Sequence
  Human HLA class I Vaccinia epitope
KLFSDISAI

SEQ ID NO: 31 - protein, 9 aa, Artificial Sequence
  Human HLA class I Vaccinia epitope
ILDDNLYKV

SEQ ID NO: 32 - protein, 9 aa, Artificial Sequence
  Human HLA class I Vaccinia epitope
KLIIHNPEL

SEQ ID NO: 33 - protein, 10 aa, Artificial Sequence
  Human HLA class I Vaccinia epitope
KITSYKFESV

SEQ ID NO: 34 - protein, 9 aa, Artificial Sequence
  Human HLA class I Vaccinia epitope
IVIEAIHTV

SEQ ID NO: 35 - protein, 9 aa, Artificial Sequence
  Human HLA class I Vaccinia epitope
GTHVLLPFY

SEQ ID NO: 36 - protein, 15 aa, Artificial Sequence
  Human HLA class I Vaccinia epitope
LRAAMISLAKKIDVQ

SEQ ID NO: 37 - protein, 9 aa, Artificial Sequence
  Human HLA class I Vaccinia epitope
VSEHFSLLF

SEQ ID NO: 38 - protein, 9 aa, Artificial Sequence
  Human HLA class I Vaccinia epitope
QSDTVFDYY

SEQ ID NO: 39 - protein, 9 aa, Artificial Sequence
  Human HLA class I Vaccinia epitope
DMCDIYLLY

SEQ ID NO: 40 - protein, 10 aa, Artificial Sequence
  Human HLA class I Vaccinia epitope
FGDSKEPVPY

SEQ ID NO: 41 - protein, 9 aa, Artificial Sequence
  Human HLA class I Vaccinia epitope
LSDLKKTIY

SEQ ID NO: 42 - protein, 9 aa, Artificial Sequence
  Human HLA class I Vaccinia epitope
ITDFNIDTY

SEQ ID NO: 43 - protein, 10 aa, Artificial Sequence
  Human HLA class I Vaccinia epitope
AVKDVTITKK

SEQ ID NO: 44 - protein, 9 aa, Artificial Sequence
  Human HLA class I Vaccinia epitope
KVMFVIRFK

SEQ ID NO: 45 - protein, 9 aa, Artificial Sequence
  Human HLA class I Vaccinia epitope
TYNDHIVNL

SEQ ID NO: 46 - protein, 9 aa, Artificial Sequence
  Human HLA class I Vaccinia epitope
VWINNSWKF

SEQ ID NO: 47 - protein, 9 aa, Artificial Sequence
  Human HLA class I Vaccinia epitope
KYQSPVNIF

SEQ ID NO: 48 - protein, 9 aa, Artificial Sequence
  Human HLA class I Vaccinia epitope
DTRGIFSAY

SEQ ID NO: 49 - protein, 9 aa, Artificial Sequence
  Human HLA class I Vaccinia epitope
VYINHPFMY

SEQ ID NO: 50 - protein, 9 aa, Artificial Sequence
  Human HLA class I Vaccinia epitope
TVADVRHCL

SEQ ID NO: 51 - protein, 9 aa, Artificial Sequence
  Human HLA class I Vaccinia epitope
MPAYIRNTL

SEQ ID NO: 52 - protein, 9 aa, Artificial Sequence
  Human HLA class I Vaccinia epitope
HPRHYATVM

SEQ ID NO: 53 - protein, 10 aa, Artificial Sequence
  Human HLA class I Vaccinia epitope
KPKPAVRFAI

SEQ ID NO: 54 - protein, 10 aa, Artificial Sequence
  Human HLA class I Vaccinia epitope
RPMSLRSTII

SEQ ID NO: 55 - protein, 9 aa, Artificial Sequence
  Human HLA class I Vaccinia epitope
APNPNRFVI

SEQ ID NO: 56 - protein, 9 aa, Artificial Sequence
  Human HLA class I Vaccinia epitope
WLKIKRDYL

SEQ ID NO: 57 - protein, 9 aa, Artificial Sequence
  Human HLA class I Vaccinia epitope
SIIDLIDEY

SEQ ID NO: 58 - protein, 15 aa, Artificial Sequence
  Human HLA class I Vaccinia epitope
CIDGKWNPILPTCVR

SEQ ID NO: 59 - protein, 9 aa, Artificial Sequence
  Human HLA class I Vaccinia epitope
SEVKFKYVL

SEQ ID NO: 60 - protein, 15 aa, Artificial Sequence
  Human HLA class I Vaccinia epitope
TKYFRCEEKNGNTSW

SEQ ID NO: 61 - protein, 9 aa, Artificial Sequence
  Human HLA class I Vaccinia epitope
DEIKCPNLN

SEQ ID NO: 62 - protein, 9 aa, Artificial Sequence
  Human HLA class I Vaccinia epitope
HDVYGVSNF

SEQ ID NO: 63 - protein, 9 aa, Artificial Sequence
  Human HLA class I Vaccinia epitope
DEVASTHDW

SEQ ID NO: 64 - protein, 9 aa, Artificial Sequence
  Human HLA class I Vaccinia epitope
YEFRKVKSY

SEQ ID NO: 65 - protein, 9 aa, Artificial Sequence
  Human HLA class I Vaccinia epitope
EERHIFLDY

SEQ ID NO: 66 - protein, 9 aa, Artificial Sequence
  Human HLA class I Vaccinia epitope
LENGAIRIY

SEQ ID NO: 67 - protein, 9 aa, Artificial Sequence
  Human HLA class I Vaccinia epitope
EEIPDFAFY

SEQ ID NO: 68 - protein, 9 aa, Artificial Sequence
  Human HLA class I Vaccinia epitope
DDVSREKSM

SEQ ID NO: 69 - protein, 9 aa, Artificial Sequence
  Human HLA class I Vaccinia epitope
IEGELESLS

SEQ ID NO: 70 - protein, 9 aa, Artificial Sequence
  Human HLA class I Vaccinia epitope
AELTIGVNY